SEQUENCE LISTING (6 sequences)

SEQ ID NO: 1 - DNA, 20 bp, Artificial
  Primer
accctatgctgctcctgcta

SEQ ID NO: 2 - DNA, 20 bp, Artificial
  primer
ggaggggatcagtgtcagaa

SEQ ID NO: 3 - DNA, 20 bp, Artificial
  primer
accacttcacaagtcggagg

SEQ ID NO: 4 - DNA, 20 bp, Artificial
  primer
attccaagaaaccatctggc

SEQ ID NO: 5 - DNA, 20 bp, Artificial
  primer
tgttaccaactgggacgaca

SEQ ID NO: 6 - DNA, 20 bp, Artificial
  primer
gtggacagtgaggccaagat

(19) United States
(12) Patent Application Publication
(10) Pub. No.: US 2009/0233999 A1
(43) Pub. Date: Sep. 17, 2009

(54) COMPOSITIONS AND THERAPEUTIC METHODS INVOLVING ISOFLAVONES AND ANALOGUES THEREOF

(75) Inventors: ANDREW HEATON, ABBOTSFORD (AU); NARESH KUMAR, MAROUBRA (AU); GRAHAM EDMUND KELLY, WAHROONGA (AU); ALAN HUSBAND, MCMAHON'S POINT (AU)

Correspondence Address: SUGHRUE MION, PLLC, 2100 PENNSYLVANIA AVENUE, N.W., SUITE 800, WASHINGTON, DC, 20037, US

(73) Assignee: NOVOGEN RESEARCH PTY LTD, NORTH RYDE (AU)

(21) Appl. No.: 12/266,449

(22) Filed: Nov. 6, 2008

Related U.S. Application Data
(63) Continuation-in-part of application No. 11/300,976, filed on Dec. 14, 2005.
Application No. 11/300,976 is a continuation of application No. 10/704,385, filed on Nov. 7, 2003, now Pat. No. 7,488,494.
Application No. 10/704,385 is a continuation of application No. 10/070,361, filed on Jul. 8, 2002, now abandoned.

(30) Foreign Application Priority Data
Sep. 6, 1999 (AU) PQ 2661
Sep. 6, 2000 (AU) PCT/AU00/01056

(51) Int. Cl. A61K 31/35; A61P 19/02

(52) U.S. Cl. 514/456

(57) ABSTRACT

Isoflavone compounds and analogues thereof, compositions containing same and therapeutic methods of treatment involving same are described.

This is a continuation-in-part of U.S. patent application Ser. No. 11/300,976, filed Dec. 14, 2005, which is a continuation of U.S. patent application Ser. No. 10/704,385, filed Nov. 7, 2003, which is a continuation of U.S. patent application Ser. No. 10/070,361, filed Jul. 8, 2002, now abandoned, which entered the U.S. National Stage under 35 U.S.C. § 371 based on PCT/AU00/01056, filed Sep. 6, 2000, which claims the benefit of Australian Application No. PQ 2661, filed Sep. 6, 1999, each of which are hereby incorporated by reference.

This invention relates to compounds, formulations, drinks, foodstuffs, methods and therapeutic uses involving, containing, comprising, including and/or for preparing certain isoflavone compounds and analogues thereof.

According to an aspect of this invention there is provided isoflavone compounds and analogues thereof of the general formula I:

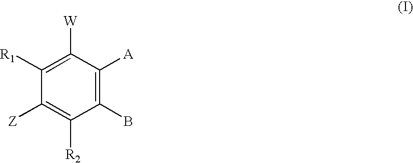

in which
R₁ and R₂ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, aryl, arylalkyl, thio, alkylthio, amino, alkylamino, dialkylamino, nitro or halo,
Z is hydrogen, and
W is R₁, A is hydrogen, hydroxy, NR₃R₄ or thio, and B is selected from

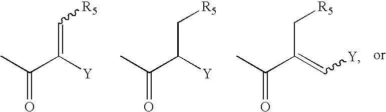

W is R₁, and A and B taken together with the carbon atoms to which they are attached form a six-membered ring selected from

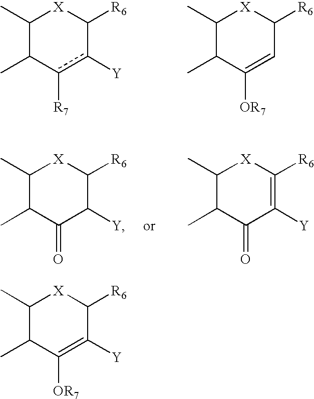

W, A and B taken together with the groups to which they are associated comprise

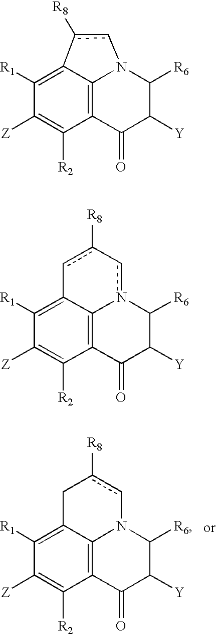

W and A taken together with the groups to which they are associated comprise

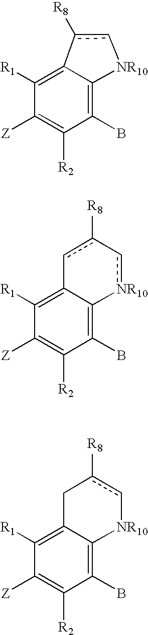

and B is

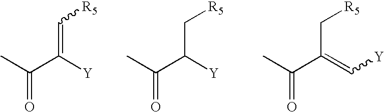

wherein
R₃ is hydrogen, alkyl, aryl, arylalkyl, an amino acid, C(O)R₁₁ where R₁₁ is hydrogen alkyl, aryl, arylalkyl or an amino acid, or CO₂R₁₂ where R₁₂ is hydrogen, alkyl, haloalkyl, aryl or arylalkyl,
R₄ is hydrogen, alkyl or aryl,
or R₃ and R₄ taken together with the nitrogen to which they are attached comprise pyrrolidinyl or piperidinyl,
R₅ is hydrogen, C(O)R₁₁ where R₁₁ is as previously defined, or CO₂R₁₂ where R₁₂ is as previously defined,
R₆ is hydrogen, hydroxy, alkyl, aryl, amino, thio, NR₃R₄, COR₁₁ where R₁₁ is as previously defined, CO₂R₁₂ where R₁₂ is as previously defined or CONR₃R₄,
R₇ is hydrogen, C(O)R₁₁ where R₁₁ is as previously defined, alkyl, haloalkyl, aryl, arylalkyl or Si(R₁₃)₃ where each R₁₃ is independently hydrogen, alkyl or aryl,
R₈ is hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀ is hydrogen, alkyl, haloalkyl, amino, aryl, arylalkyl, an amino acid, alkylamino or dialkylamino,
the drawing [symbol] represents either a single bond or a double bond,
X is O, NR₄ or S, and
Y is

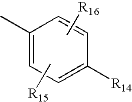

wherein
R₁₄, R₁₅ and R₁₆ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, aryl, arylalkyl, thio, alkylthio, amino, alkylamino, dialkylamino, nitro or halo, with the proviso that when
R₁ is hydroxy, or OC(O)R_{A} where R_{A} is alkyl or an amino acid, and
R₂ is hydrogen, hydroxy, OR_{B} where R_{B} is an amino acid or C(O)R_{A} where R_{A} is as previously defined, and
W is hydrogen, then
Y is not phenyl, 4-hydroxyphenyl, 4-acetoxyphenyl, 4-alkoxyphenyl or 4-alkylphenyl.

According to another aspect of this invention there is provided isoflavone compounds and analogues thereof of the general formula II:

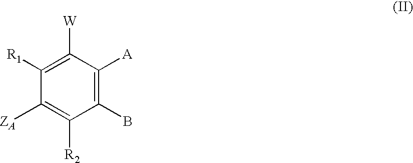

in which
R₁ and R₂ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, aryl, arylalkyl, thio, alkylthio, amino, alkylamino, dialkylamino, nitro or halo,
Z_{A} is OR₉, OC(O)R₁₀, OS(O)R₁₀, CHO, C(O)R₁₀, COOH, CO₂R₁₀, CONR₃R₄, alkyl, haloalkyl, aryl, arylalkyl, thio, alkylthio, amino, alkylamino, dialkylamino, nitro or halo, and
W is R₁, A is hydrogen, hydroxy, NR₃R₄ or thio, and B is selected from

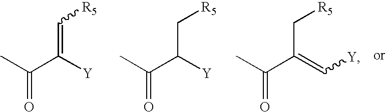

W is R₁, and A and B taken together with the carbon atoms to which they are attached form a six-membered ring selected from

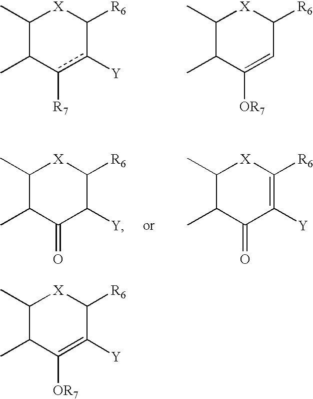

W, A and B taken together with the groups to which they are associated comprise

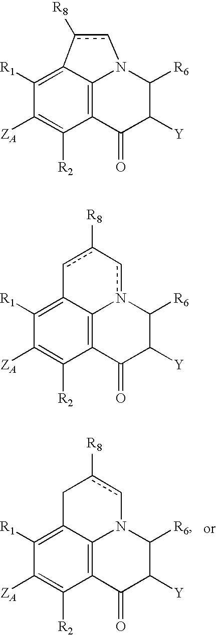

W and A taken together with the groups to which they are associated comprise

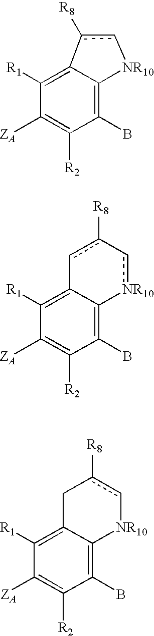

and B is

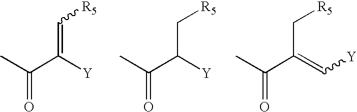

wherein
R₃ is hydrogen, alkyl, aryl, arylalkyl, an amino acid, C(O)R₁₁ where R₁₁ is hydrogen alkyl, aryl, arylalkyl or an amino acid, or CO₂R₁₂ where R₁₂ is hydrogen, alkyl, haloalkyl, aryl or arylalkyl,
R₄ is hydrogen, alkyl or aryl,
or R₃ and R₄ taken together with the nitrogen which they are attached are pyrrolidinyl or piperidinyl,
R₅ is hydrogen, C(O)R₁₁ where R₁₁ is as previously defined, or CO₂R₁₂ where R₁₂ is as previously defined,
R₆ is hydrogen, hydroxy, alkyl, aryl, amino, thio, NR₃R₄, COR₁₁ where R₁₁ is as previously defined, CO₂R₁₂ where R₁₂ is as previously defined or CONR₃R₄,
R₇ is hydrogen, C(O)R₁₁ where R₁₁ is as previously defined, alkyl, haloalkyl, aryl, arylalkyl or Si(R₁₃)₃ where each R₁₃ is independently hydrogen, alkyl or aryl,
R₈ is hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀ is hydrogen, alkyl, haloalkyl, amino, aryl, arylalkyl, an amino acid, alkylamino or dialkylamino,
the drawing [symbol] represents either a single bond or a double bond,
X is O, NR₄ or S, and
Y is

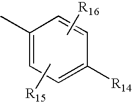

wherein
R₁₄, R₁₅ and R₁₆ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, aryl, arylalkyl, thio, alkylthio, amino, alkylamino, dialkylamino, nitro or halo.

It has surprisingly been found by the inventors that compounds of the general formulae I and II:

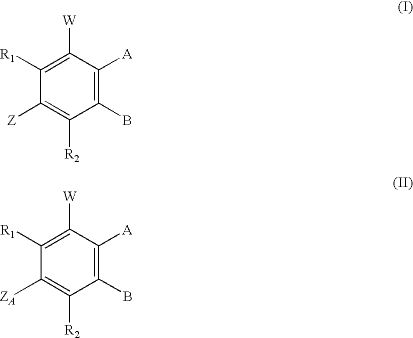

in which
R₁, R₂, W, A, B, Z and Z_{A} are as defined above have particular utility and effectiveness in the treatment, prophylaxis, amelioration defense against, and/or prevention of menopausal syndrome including hot flushes, anxiety, depression, mood swings, night sweats, headaches, and urinary incontinence; osteoporosis; premenstrual syndrome, including fluid retention, cyclical mastalgia, and dysmenorrhoea; Reynaud's Syndrome; Reynaud's Phenomenon; Buergers Disease; coronary artery spasm; migraine headaches; hypertension; benign prostatic hypertrophy; all forms of cancer including breast cancer; uterine cancer; ovarian cancer; testicular cancer; large bowel cancer; endometrial cancer; prostatic cancer; uterine cancer; atherosclerosis; Alzheimers disease; inflammatory diseases including inflammatory bowel disease, ulcerative colitis, Crohns disease; rheumatic diseases including rheumatoid arthritis; acne; baldness including male pattern baldness (alopecia hereditaria); psoriasis; diseases associated with oxidant stress including cancer; myocardial infarction; stroke; arthritis; sunlight induced skin damage or cataracts.

Thus according to another aspect of the present invention there is provided a method for the treatment, prophylaxis, amelioration, defense against, and/or prevention of menopausal syndrome including hot flushes, anxiety, depression, mood swings, night sweats, headaches, and urinary incontinence; osteoporosis; premenstrual syndrome, including fluid retention, cyclical mastalgia, and dysmenorrhoea; Reynaud's Syndrome; Reynaud's Phenomenon; Buergers Disease; coronary artery spasm; migraine headaches; hypertension; benign prostatic hypertrophy; all forms of cancer including breast cancer; uterine cancer; ovarian cancer; testicular cancer; large bowel cancer; endometrial cancer; prostatic cancer; uterine cancer; artherosclerosis; Alzheimers disease; inflammatory diseases including inflammatory bowel disease, ulcerative colitis, Crohns disease; rheumatic diseases including rheumatoid arthritis; acne; baldness including male pattern baldness (alopecia hereditaria); psoriasis; diseases associated with oxidant stress including cancer; myocardial infarction; stroke; arthritis; sunlight induced skin damage or cataracts (for convenience hereafter referred to as the “therapeutic indications”) which comprises administering to a subject a therapeutically effective amount of one or more compounds of formulae I and II as defined above.

Yet another aspect of the present invention is the use of compounds of formulae I and II for the manufacture of a medicament for the treatment, amelioration, defense against, prophylaxis and/or prevention of one or more of the therapeutic indications.

Still another aspect of the present invention is the use of one or more compounds of formulae I and II in the treatment, amelioration, defense against, prophylaxis and/or prevention of one or more of the therapeutic indications.

And another aspect of the present invention comprises an agent for the treatment, prophylaxis, amelioration, defense against and/or treatment of the therapeutic indications which comprises one or more compounds of formulae I and II either alone or in association with one or more carriers or excipients.

A further aspect of the invention is a therapeutic composition which comprises one or more compounds of formulae I and II in association with one or more pharmaceutical carriers and/or excipients.

A still further aspect of the present invention is a drink or food-stuff, which contains one or more compounds of formulae I and II.

Another aspect of the present invention is a microbial culture or a food-stuff containing one or more microbial strains which microorganisms produce one or more compounds of formulae I and II.

Still another aspect of the present invention relates to one or more microorganisms which produce one or more compounds of formulae I and II. Preferably the microorganism is a purified culture, which may be admixed and/or administered with one or more other cultures which product compounds of formulae I and II.

The invention subject of this continuation-in-part application specifically relates to a method for the treatment or prophylaxis of an inflammatory disease or a disease associated with oxidant stress which comprises the step of administering to a subject a therapeutically effective amount of one or more compounds of the general formula:

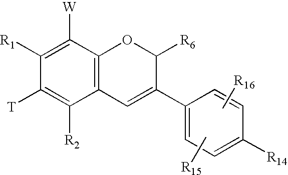

in which
R₁ is hydroxy or OC(O)R₁₀,
R₂ is hydrogen, hydroxy, OR₉, OC(O)R₁₀, alkyl or halo,
T is hydrogen, alkyl or halo,
W is hydrogen, hydroxy, OC(O)R₁₀, alkyl or halo,
R₆ is hydrogen,
R₉ is alkyl,
R₁₀ is hydrogen or alkyl,
R₁₄, R₁₅ and R₁₆ are independently hydrogen, hydroxy, OR₉, OC(O)R₁₀ or halo,
   or a pharmaceutically acceptable salt thereof,
   with the proviso that
   when
R₁ is hydroxy or OC(O)R_{A} where R_{A} is alkyl, and
R₂ is hydrogen, hydroxy, OR_{B} where R_{B} is C(O)R_{A} where R_{A} is alkyl,
W is hydrogen, and
T is hydrogen, then
Y is not phenyl, 4-hydroxyphenyl, 4-acetoxyphenyl, 4-alkoxyphenyl or 4-alkylphenyl; and
   with the proviso that the following compounds are excluded:

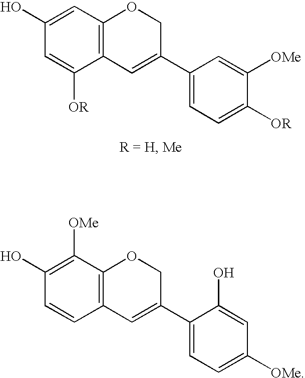

These and other aspects and embodiments of the invention are set out below and the claims that follow.

Throughout this specification and the claims which follow, unless the text requires otherwise, the word “comprise”, and variations such as “comprises” or “comprising”, will be understood to imply the inclusion of a stated integer or step or group of integers or steps but not the exclusion of any other integer or step or group of integers or steps.

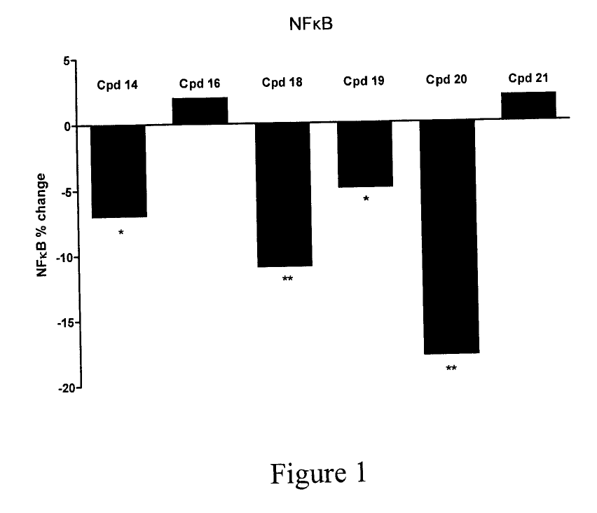

FIG. 1 shows the mean change in NFκB promoter activity in THP-1 cells by test compounds at 30 μM relative to treatment with vehicle alone.

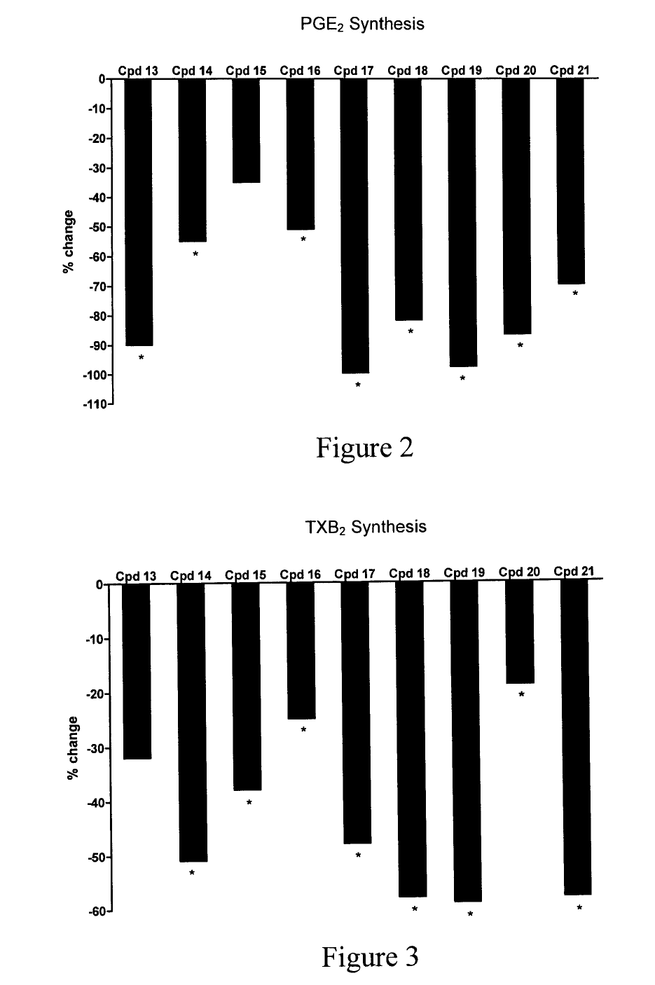

FIG. 2 shows the mean change of LPS-induced PGE₂ synthesis in human monocytes by test compounds relative to treatment with vehicle alone.

FIG. 3 shows the mean change of LPS-induced TXB2 synthesis in human monocytes by test compounds relative to treatment with vehicle alone.

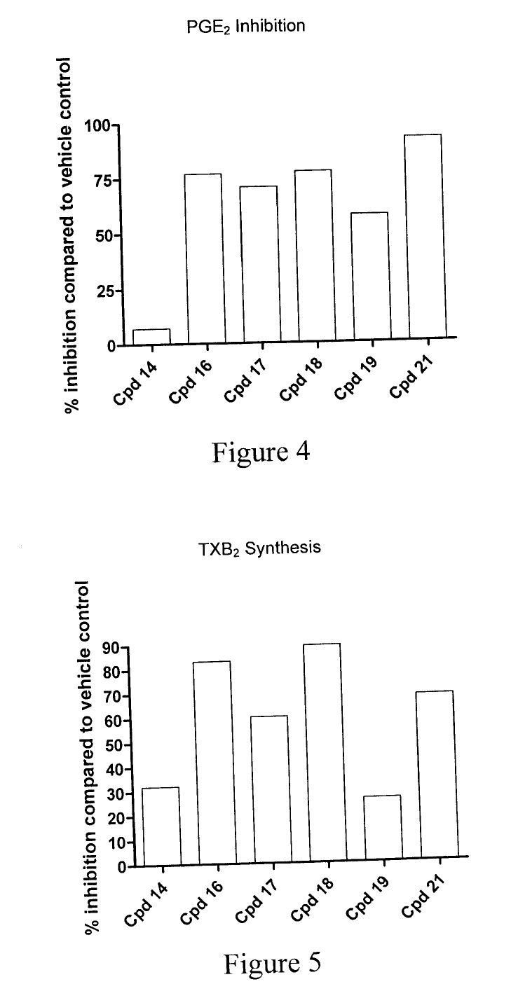

FIG. 4 shows the mean change of LPS-induced PGE2 synthesis in RAW 264.7 murine macrophages by test compounds at 1 μM relative to treatment with vehicle alone.

FIG. 5 shows the mean change of LPS-induced TXB2 synthesis in RAW 264.7 murine macrophages by test compounds at 1 μM relative to treatment with vehicle alone.

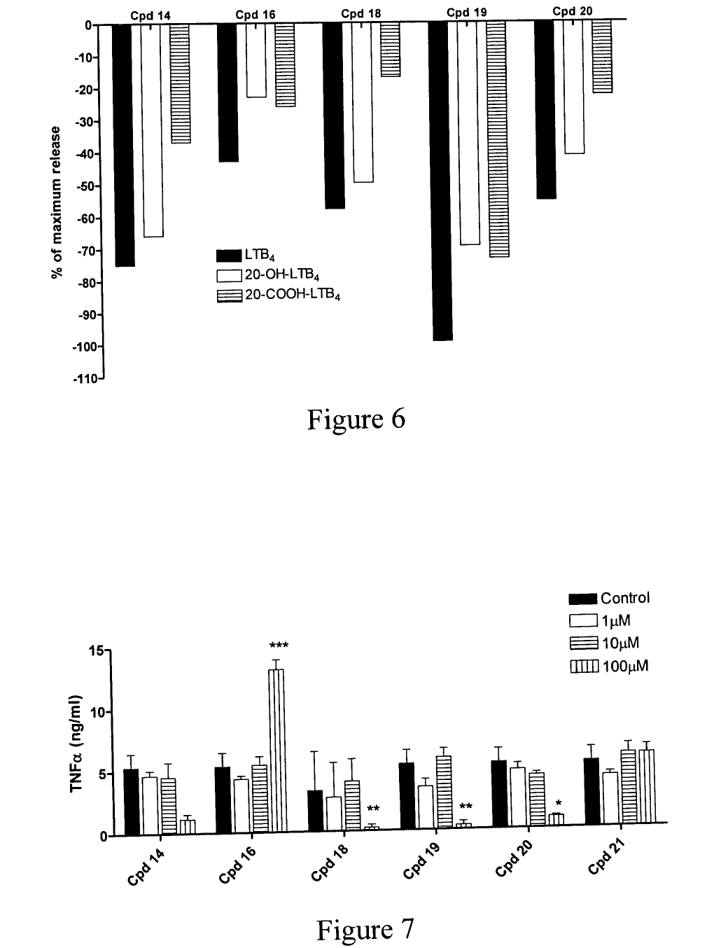

FIG. 6 shows the effect of test compounds on synthesis of LTB4, 20-OH-LTB4 and 20-COOH-LTB4 at 1 μM.

FIG. 7 shows the effect of LPS-induced TNFα synthesis in human monocytes by test compounds relative to treatment with vehicle alone.

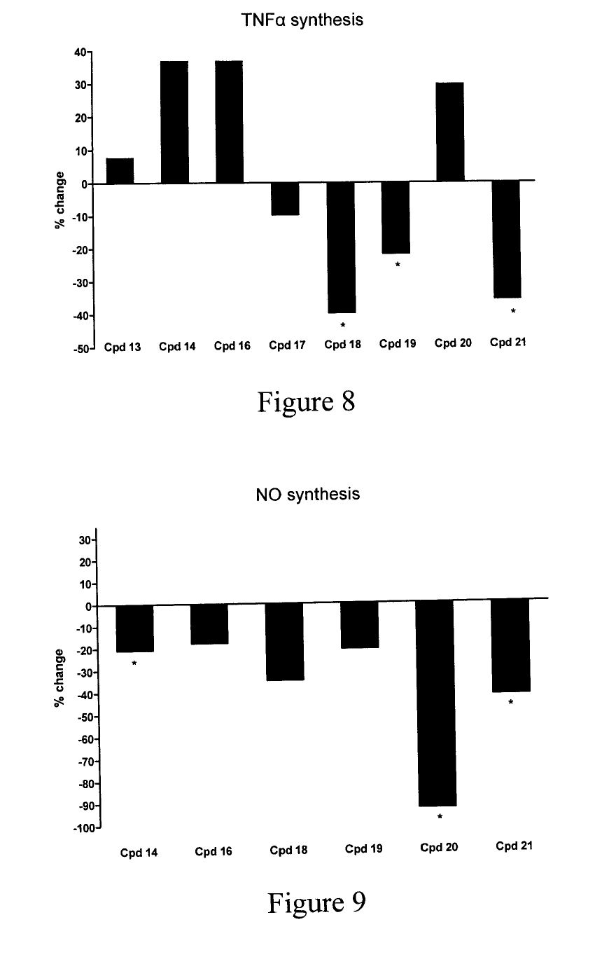

FIG. 8 shows the mean change of LPS-induced TNFα synthesis RAW 264.7 murine macrophages by test compounds relative to treatment with vehicle alone.

FIG. 9 shows the mean change of LPS-induced NO synthesis in RAW 264.7 murine macrophages by test compounds relative to treatment with vehicle alone.

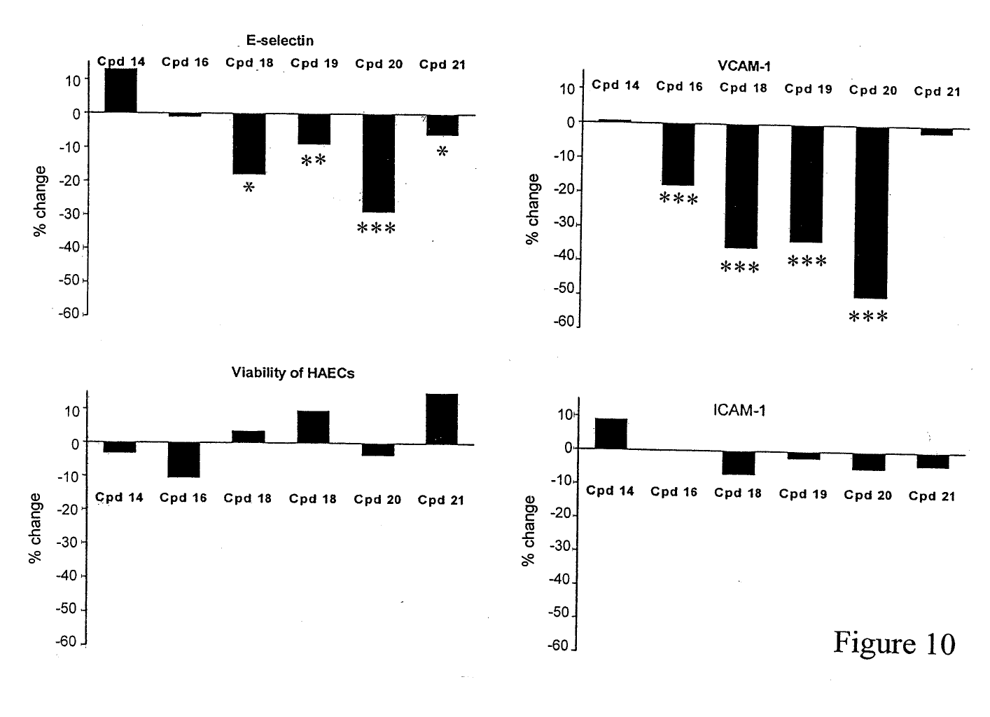

FIG. 10 shows the effect on the expression of TNFα-induced VCAM-1, ICAM-1 and E-selection, and cell viability of HAECs following incubation with 10 μM of test compound.

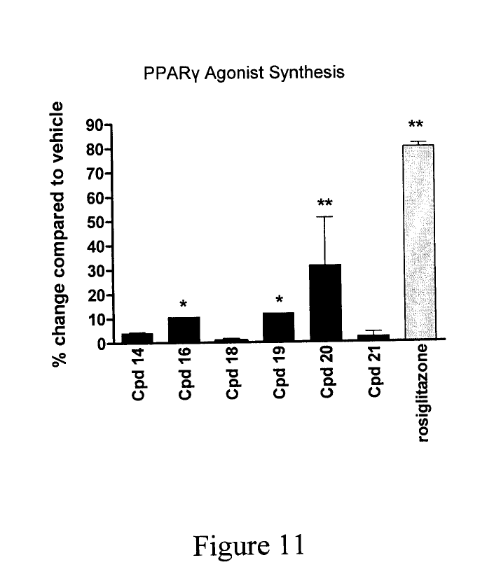

FIG. 11 shows the PPARγ agonist activity of test compounds at 5 μM.

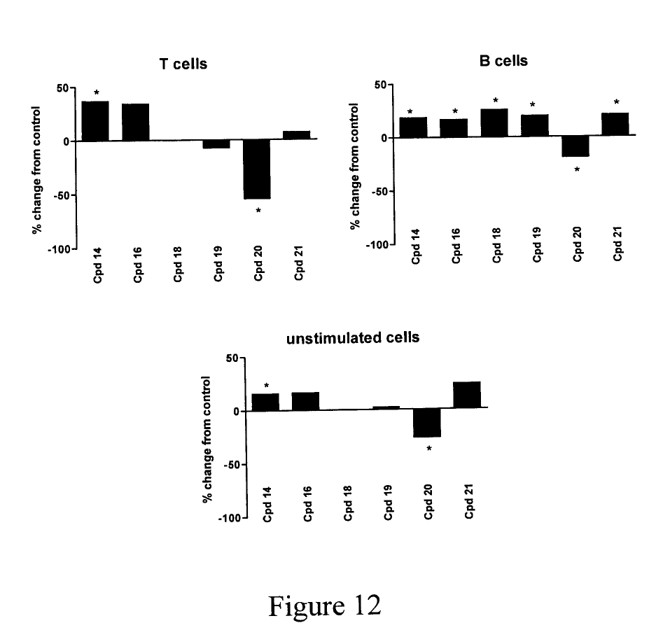

FIG. 12 shows the effect of test compounds on murine splenocyte proliferation.

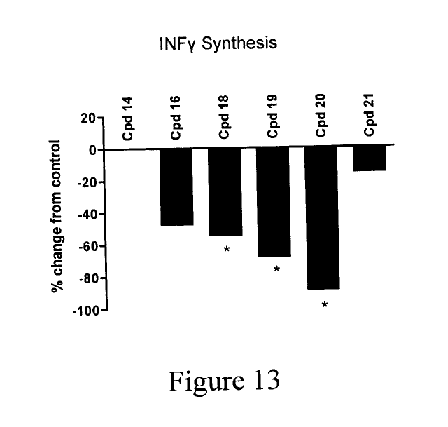

FIG. 13 shows the effect of test compounds on the synthesis of INFγ.

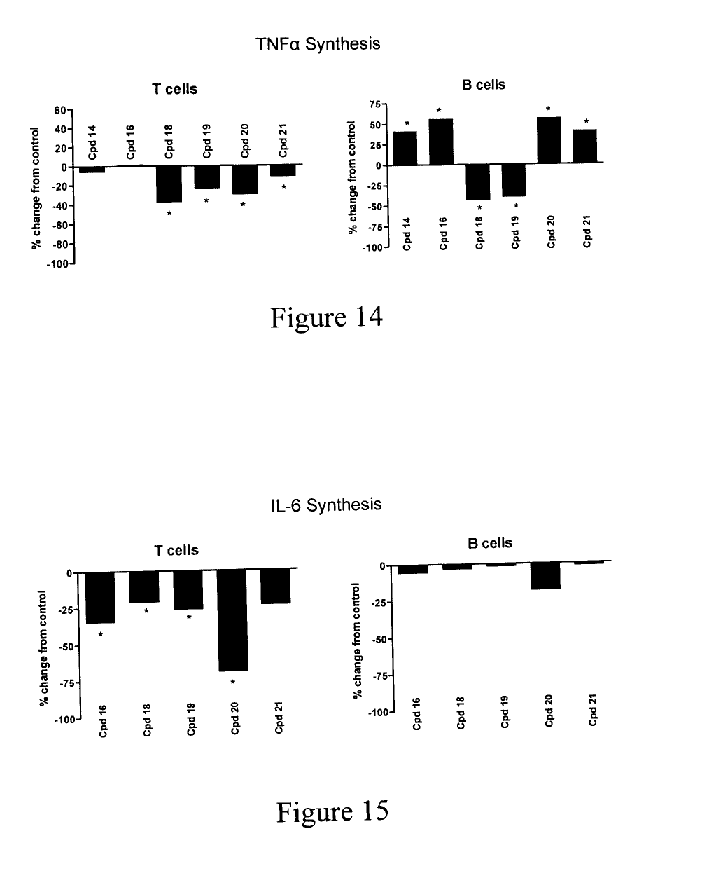

FIG. 14 shows the effect of test compounds on the synthesis of TNFα.

FIG. 15 shows the effect of test compounds on the synthesis of IL-6.

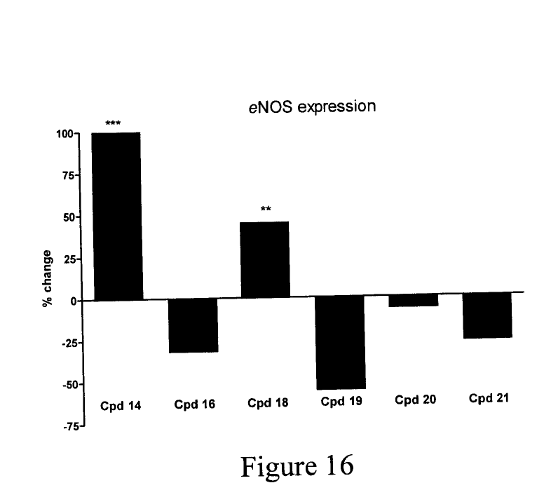

FIG. 16 shows the effect on the expression eNOS and viability in HAECs following incubation with 10 μM test compound.

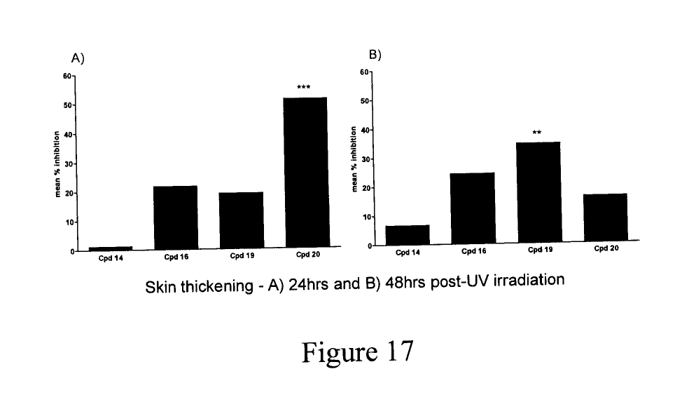

FIG. 17 shows the mean percentage inhibition of UV-induced skin thickening by test compounds relative to treatment with vehicle alone at 24 hrs (A) and 48 hrs (B) post-UV irradiation.

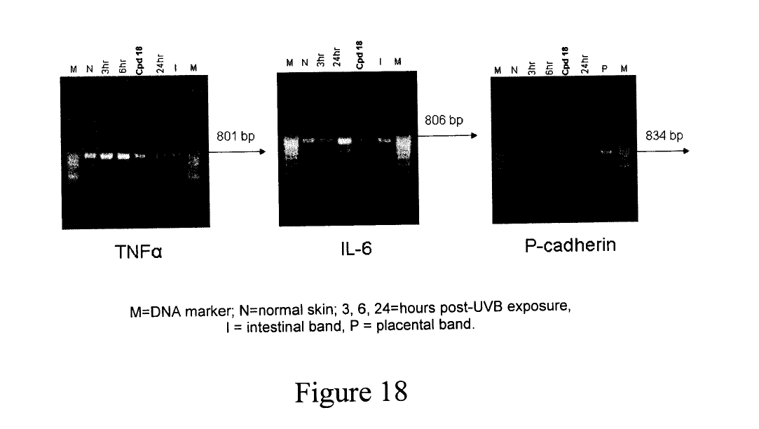

FIG. 18 shows the RT-PCR amplification of TNFα, IL-6 and P-cadherin mRNAs extracted from C3H/HeN (for TNF-α) or Skh:hr-1 skin. (M=DNA marker; N=normal skin; 3, 6, 24=hours post-UVB exposure, I=intestinal band, P=placental band).

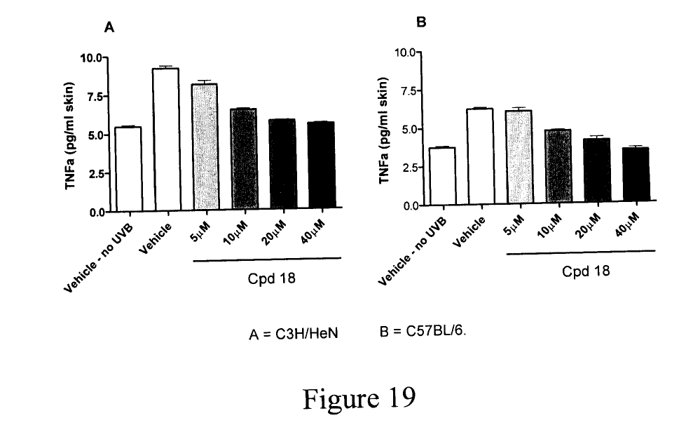

FIG. 19 shows the UVB-induced TNF-α protein released from skin at 3 h post-irradiation with test compounds.

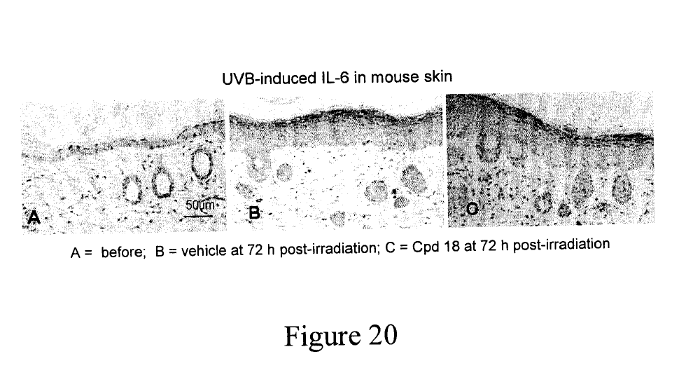

FIG. 20 shows the immunohistochemical identification of UVB-induced IL-6 in mouse skin with Cpd. 18, where A=before; B=vehicle at 72 h post-irradiation; C=Cpd. 18 at 72 h post-irradiation.

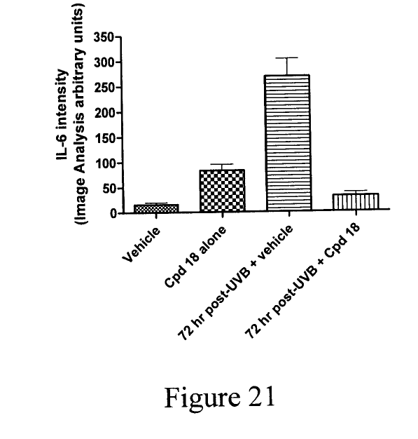

FIG. 21 shows the semi-quantitation by image analysis of the average staining intensity with Cpd. 18, where Mean±SEM, n=15 sequential fields, ×20 magnification, 3 mice per group.

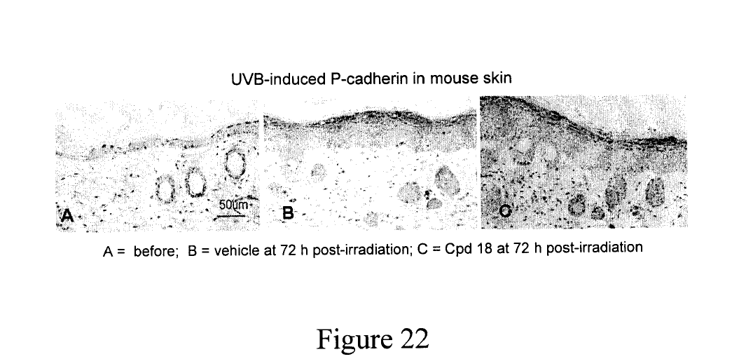

FIG. 22 shows the immunohistochemical identification of UVB-induced P-cadherin in mouse skin with Cpd. 18, where A=before; B=vehicle at 72 h post-irradiation; C=Cpd. 18 at 72 h post-irradiation.

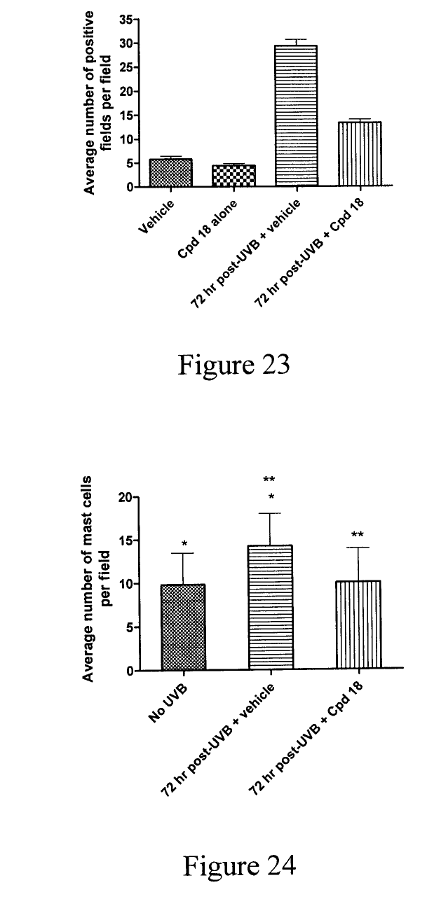

FIG. 23 shows the semi-quantitation by image analysis of the average staining intensity with Cpd. 18, where Mean±SEM, n=10 sequential fields, ×20 magnification, 3 mice per group.

FIG. 24 shows the average mast cell number before and post-UVB treatment for Cpd. 18, where Mean±SD, n=30 fields, ×40 magnification, 3 mice per group.

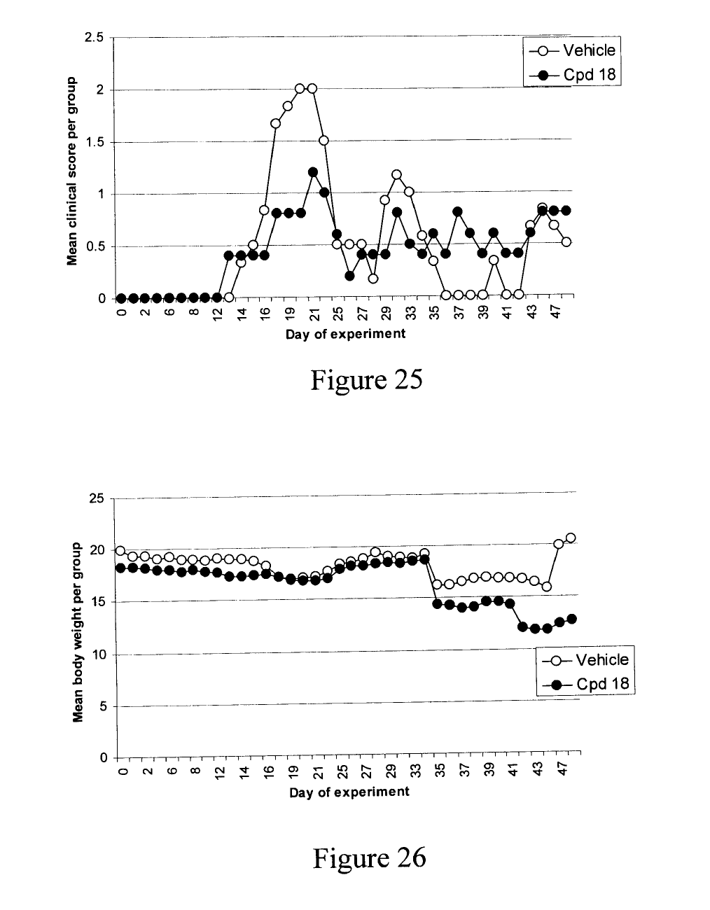

FIG. 25 shows the average clinical score in murine EAE model with Cpd. 18.

FIG. 26 shows the average body weight in murine EAE model with Cpd. 18.

The term “alkyl” is taken to mean both straight chain and branched chain alkyl groups such as methyl, ethyl, propyl, isopropyl, butyl, isobutyl, secbutyl, tertiary butyl, and the like. The alkyl group has 1 to 10 carbon atoms, preferably from 1 to 6 carbon atoms, more preferably methyl, ethyl, propyl or isopropyl. The alkyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “aryl” is taken to include phenyl and naphthyl and may be optionally substituted by one or more C₁-C₄-alkyl, hydroxy, C₁-C₄-alkoxy, carbonyl, C₁-C₄-alkoxycarbonyl, C₁-C₄-alkylcarbonyloxy or halo.

The term “halo” is taken to include fluoro, chloro, bromo and iodo, preferably fluoro and chloro, more preferably fluoro. Reference to for example “haloalkyl” will include monohalogenated, dihalogenated and up to perhalogenated alkyl groups. Preferred haloalkyl groups are trifluoromethyl and pentafluoroethyl.

Particularly preferred compounds of the present invention are selected from:

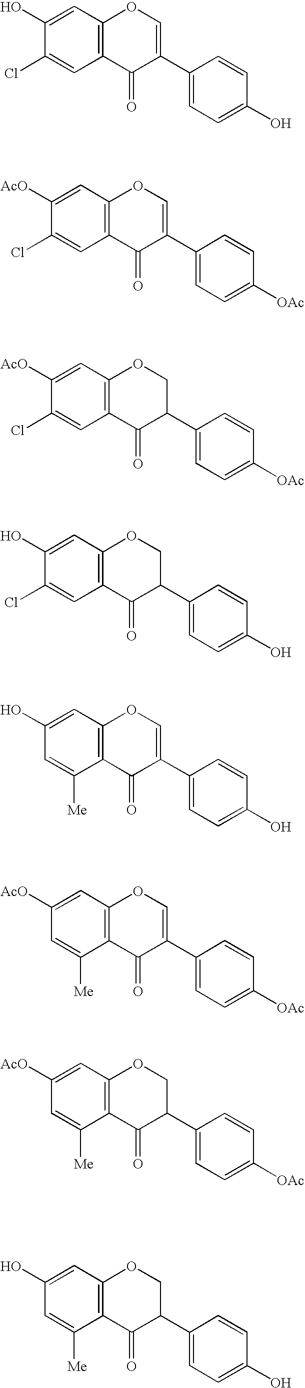

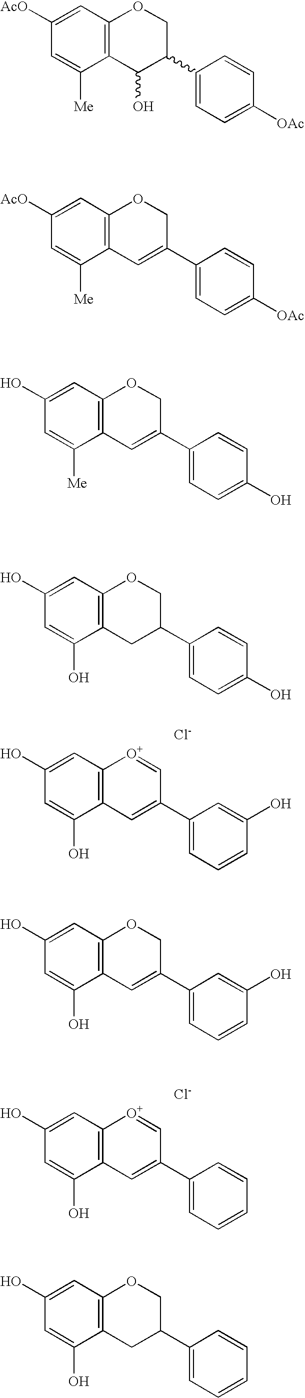

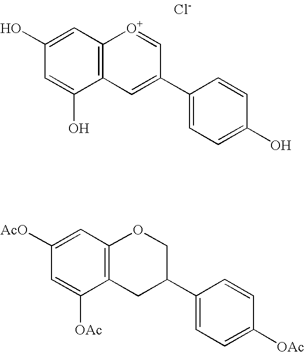

Compounds of the present invention have particular application in the treatment of diseases associated with or resulting from estrogenic effects, androgenic effects, vasodilatory and spasmodic effects, inflammatory effects and oxidative effects.

The amount of one or more compounds of formulae I and II which is required in a therapeutic treatment according to the invention will depend upon a number of factors, which include the specific application, the nature of the particular compound used, the condition being treated, the mode of administration and the condition of the patient. Compounds of formulae I or II may be administered in a manner and amount as is conventionally practised. See, for example, Goodman and Gilman, The Pharmacological Basis of Therapeutics, 1299 (7th Edition, 1985). The specific dosage utilised will depend upon the condition being treated, the state of the subject, the route of administration and other well known factors as indicated above. In general, a daily dose per patient may be in the range of 0.1 mg to 2 g; typically from 0.5 mg to 1 g; preferably from 50 mg to 200 mg.

The production of pharmaceutical compositions for the treatment of the therapeutic indications herein described are typically prepared by admixture of the compounds of the invention (for convenience hereafter referred to as the “active compounds”) with one or more pharmaceutically or veterinarially acceptable carriers and/or excipients as are well known in the art.

The carrier must, of course, be acceptable in the sense of being compatible with any other ingredients in the formulation and must not be deleterious to the subject. The carrier or excipient may be a solid or a liquid, or both, and is preferably formulated with the compound as a unit-dose, for example, a tablet, which may contain from 0.5% to 59% by weight of the active compound, or up to 100% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, optical, buccal (for example, sublingual), parenteral (for example, subcutaneous, intramuscular, intradermal, or intravenous) and transdermal administration, although the most suitable route in any given case will depend on the nature and severity of the condition being treated and on the nature of the particular active compound which is being used.

Formulation suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more accessory ingredients. Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Formulations suitable for buccal (sublingual) administration include lozenges comprising the active compound in a flavoured base, usually sucrose and acacia or tragacanth; and pastilles comprising the compound in an inert base such as gelatin and glycerin or sucrose and acacia.

Compositions of the present invention suitable for parenteral administration conveniently comprise sterile aqueous preparations of the active compounds, which preparations are preferably isotonic with the blood of the intended recipient. These preparations are preferably administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound and are administered at a rate of 0.1 ml/minute/kg.

Formulations suitable for rectal administration are preferably presented as unit dose suppositories. These may be prepared by admixing the active compound with one or more conventional solid carriers, for example, cocoa butter, and then shaping the resulting mixture.

Formulations or compositions suitable for topical administration to the skin preferably take the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 0.5% w/w, for example, from 0.5% to 2% w/w. Examples of such compositions include cosmetic skin creams.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound.

Formulations suitable for transdermal administration may also be delivered by iontophoresis (see, for example, Pharmaceutical Research 3 (6), 318 (1986)) and typically take the form of an optionally buffered aqueous solution of the active compound. Suitable formulations comprise citrate or bis/tris buffer (pH 6) or ethanol/water and contain from 0.1 M to 0.2 M active ingredient.

The active compounds may be provided in the form of food stuffs, such as being added to, admixed into, coated, combined or otherwise added to a food stuff. The term food stuff is used in its widest possible sense and includes liquid formulations such as drinks including dairy products and other foods, such as health bars, desserts, etc. Food formulations containing compounds of the invention can be readily prepared according to standard practices.

Compounds of the present invention have potent antioxidant activity and thus find wide application in pharmaceutical and veterinary uses, in cosmetics such as skin creams to prevent skin ageing, in sun screens, in foods, health drinks, shampoos, and the like.

It has surprisingly been found that compounds of the formulae I or II interact synergistically with vitamin E to protect lipids, proteins and other biological molecules from oxidation.

Accordingly a further aspect of this invention provides a composition comprising one or more compounds of formulae I or II, vitamin E, and optionally a pharmaceutically, veterinarially or cosmetically acceptable carriers and/or excipients.

Therapeutic methods, uses and compositions may be for administration to humans or animals, such as companion and domestic animals (such as dogs and cats), birds (such as chickens, turkeys, ducks), livestock animals (such as cattle, sheep, pigs and goats) and the like.

Compounds of formulae I and II may be prepared by standard methods known to those skilled in the art. Suitable methods may be found in, for example, International Patent Application WO 98/08503 which is incorporated herein in its entirety by reference. Methods which may be employed by those skilled in the art of chemical synthesis for constructing the general ring structures depicted in formulae I and II are depicted in schemes 1-8 below. Chemical functional group protection, deprotection, synthons and other techniques known to those skilled in the art may be used where appropriate in the synthesis of the compounds of the present invention. In the formulae depicted in the schemes below the moities R₁, R₂, R₆, R₈, R₁₄, R₁₅, R₁₆, W and X are as defined above. The moiety T is either Z or Z_{A} as defined in formulae I or II above. Reduction of the isoflavone derivatives may be effected by procedures well known to those skilled in the art including sodium borohydride reduction, and hydration over metal catalysts such as Pd/C, Pd/CaCO₃ and Platinum(IV) oxide (Adam's catalyst) in protic or aprotic solvents. The end products and isomeric ratios can be varied depending on the catalyst/solvent system chosen. The schemes depicted below are not to be considered limiting on the scope of the invention described herein.

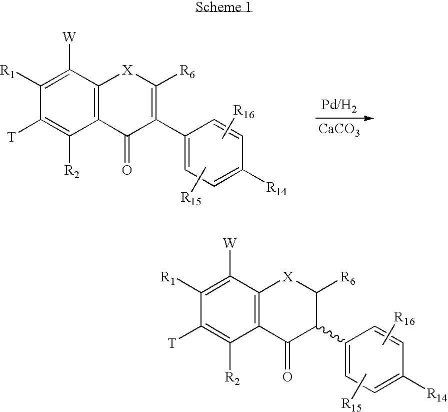

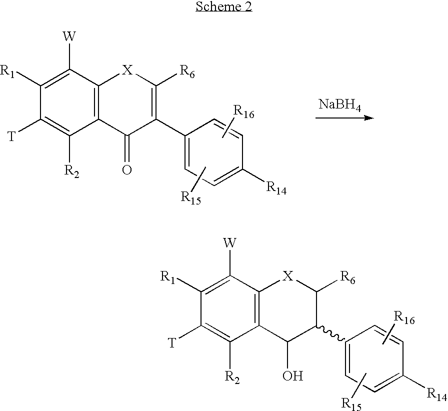

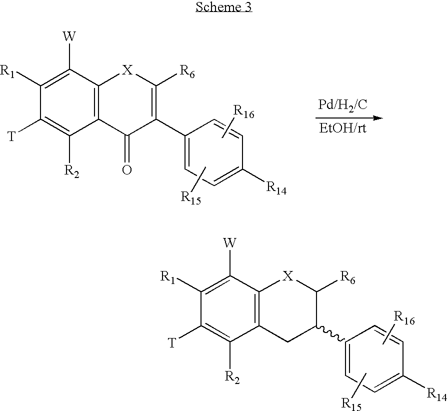

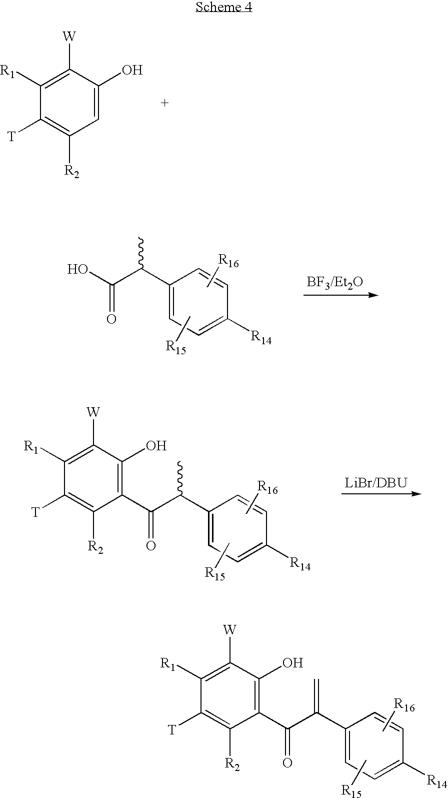

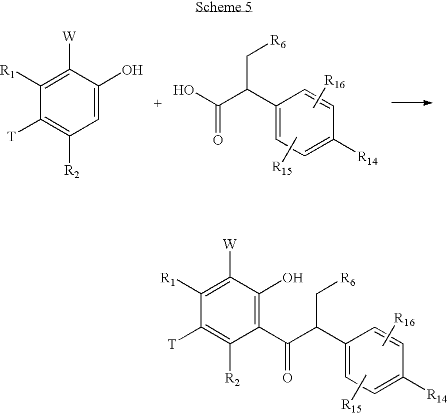

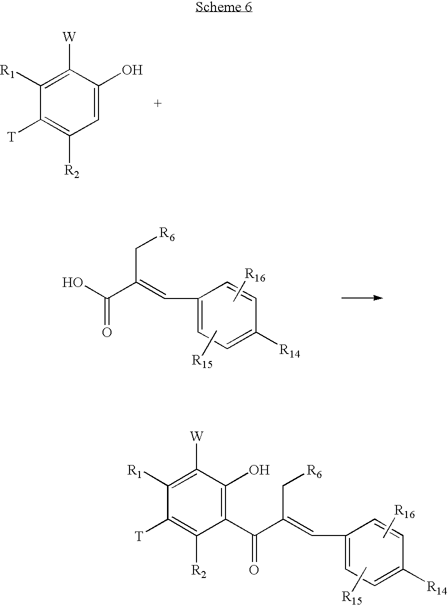

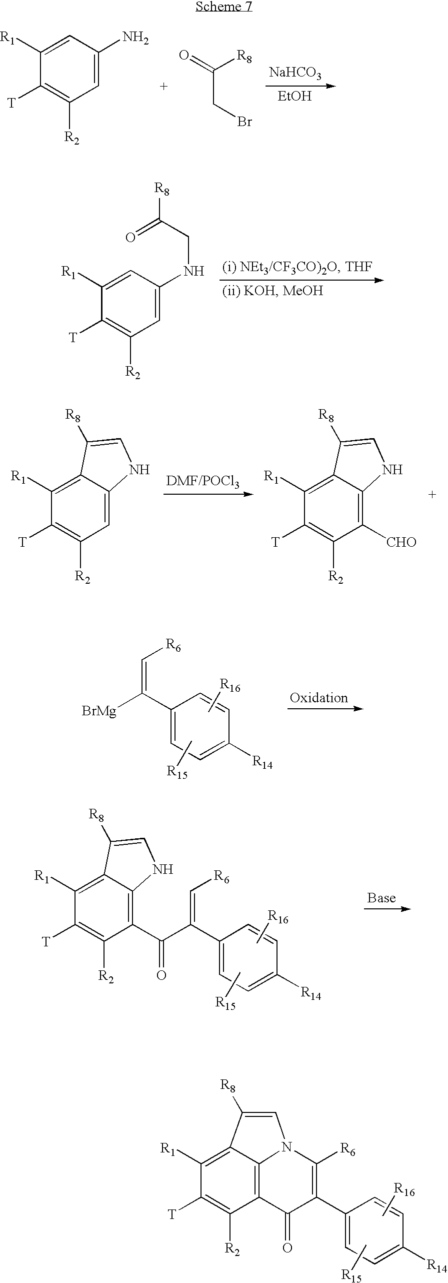

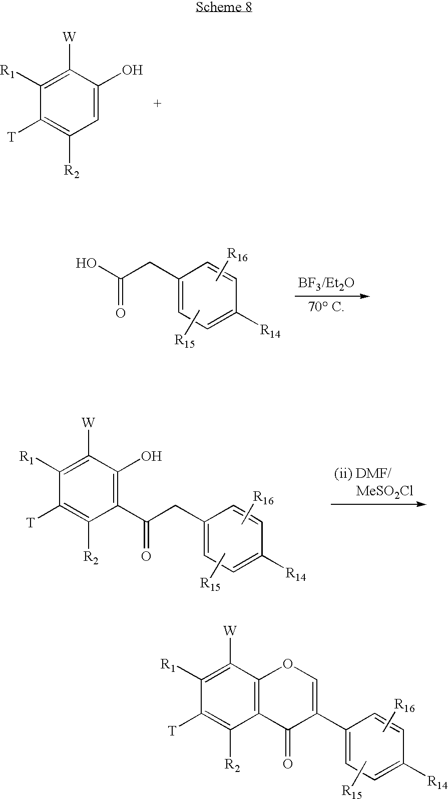

### EXAMPLE 1

### General Syntheses of Substituted Isoflavones

6-Chloro-4′,7-dihydroxyisoflavone was synthesised by the condensation of 4-chlororesorcinol with 4-hydroxyphenylacetic acid to afford 5-chloro-2,4,4′-trihydroxydeoxybenzoin. Cyclisation of the intermediate deoxybenzoin was achieved by treatment with dimethylformamide and methanesulfonyl chloride in the presence of boron triflouride etherate.

By varying the substitution pattern on the resorcinol or phenylacetic acid groups numerous other substituted isoflavones can also be synthesised in a similar manner. For example starting with 5-methyl resorcinol affords 4′,7-dihydroxy-5-methylisoflavone, whilst use of 3-hydroxy phenyl acetic acid in the general synthetic method affords 3′-hydroxy isoflavone derivatives.

Isoflavan-4-ones

### EXAMPLE 2

### Synthesis of 6-Chloro-4′,7-diacetoxyisoflavone

A mixture of 6-chloro-4′,7-dihydroxyisoflavone (1.25 g, 4.3 mmol), acetic anhydride (7.5 ml) and pyridine (1.4 ml) was heated in an oil bath at 105-110° C. for 1 h. After cooling the mixture to room temperature, it was stirred for a further 30 min during which time the diacetate crystallised from the solution. The product was filtered, washed thoroughly with aqueous methanol (50%) and dried to yield 6-chloro-4′,7-diacetoxyisoflavone (1.2 g, 75%) as colourless prisms. ¹H NMR (CDCl₃): δ 2.32 (s, 3H, OCOCH₃), 2.41 (s, 3H, OCOCH₃), 7.16 (d, 2H, J=8.6 Hz, ArH), 7.36 (s, 1H, H8), 7.57 (d, 2H, J=8.6 Hz, ArH), 8.00 (s, 1H, H5), 8.37 (s, 1H, H2).

### EXAMPLE 3

### Synthesis of 6-Chloro-4′,7-diacetoxyisoflavan-4-one

Adam's catalyst (0.045 g) was added to a solution of 6-chloro-4′,7-diacetoxyisoflavone (0.25 g, 0.7 mmol) in ethyl acetate (30 ml) and the mixture was stirred at room temperature under a hydrogen atmosphere for 24 h. The catalyst was removed by filtration through Celite and the resulting filtrate was evaporated in vacuo. The residue was recrystallised from ethanol to yield 6-chloro-4′,7-diacetoxyisoflavan-4-one (0.15 g, 60%) as colourless plates. ¹H NMR (CDCl₃): δ 2.29 (s, 3H, OCOCH₃), 2.37 (s, 3H, OCOCH₃), 3.98 (dd, 1H, J=6.0 Hz, 7.5 Hz, H3), 4.68 (m, 2H, H2), 6.87 (s, 1H, H8), 7.07 (d, 2H, J=8.6 Hz, ArH), 7.27 (d, 2H, J=8.6 Hz, ArH), 8.01 (s, 1H, H5).

### EXAMPLE 4

### Synthesis of 6-Chloro-4′,7-dihydroxyisoflavan-4-one

Imidazole (0.60 g) was added to a suspension of 6-chloro-4′,7-diacetoxyisoflavan-4-one (0.24 g, 0.06 mmol) in absolute ethanol (5.0 ml) and the mixture was refluxed for 45 min under argon. The solution was concentrated under reduced pressure and distilled water (10 ml) was added to the residue. The mixture was left overnight in the fridge and the resulting precipitate was filtered, washed with water and dried to yield 6-chloro-4′,7-dihydroxyisoflavan-4-one (0.14 g, 75%) as a white powder. ¹H NMR (d₆-acetone): δ 3.87 (t, 1H, J 7.2 Hz, H3), 4.64 (d, 2H, J 6.2 Hz, H2), 6.59 (s, 1H, H8), 6.78 (d, 2H, J 8.7 Hz, ArH), 7.10 (d, 2H, J 8.7 Hz, ArH), 7.70 (bs, 1H, OH), 7.77 (s, 1H, H5).

### EXAMPLE 5

### Synthesis of 4′,7-Diacetoxy-5-methylisoflavone

A mixture of 4′,7-dihydroxy-5-methylisoflavone (1.51 g, 5.6 mmol), acetic anhydride (9 ml) and pyridine (1.7 ml) was heated in an oil bath at 105-110° C. for 1 h. After cooling the mixture to room temperature, it was stirred for a further 30 min during which time the diacetate crystallised from the solution. The product was filtered, washed thoroughly with water and recrystallised from methanol to yield 4′,7-diacetoxy-5-methylisoflavone as colourless prisms (1.8 g, 91%). m.p. 195-97° C., ¹H NMR (CDCl₃): δ 2.32 (s, 3H, OCOCH₃), 2.35 (s, 3H, OCOCH₃), 2.87 (s, 3H, Me), 6.92 (bs, 1H, H8), 7.12 (bs, 1H, H5), 7.16 (d, 2H, J 8.7 Hz, ArH), 7.55 (d, 2H, J 8.7 Hz, ArH), 7.89 (s, 1H, H2).

### EXAMPLE 6

### Synthesis of 4′,7-Diacetoxy-5-methylisoflavan-4-one

Palladium on barium sulfate (5%, 0.06 g) was added to a solution of 4′,7-diacetoxy-5-methylisoflavone (0.30 g, 0.8 mmol) in ethyl acetate (50 ml) and the mixture was stirred at room temperature under a hydrogen atmosphere for 24 h. The catalyst was removed by filtration through Celite and the resulting filtrate was evaporated in vacuo. The residue was recrystallised from ethanol to yield 4′,7-diacetoxy-5-methylisoflavan-4-one (0.20 g, 67%) as colourless plates. m.p. 143-45° C., ¹H NMR (CDCl₃): δ 2.29 (s, 3H, OCOCH₃), 2.30 (s, 3H, OCOCH₃), 2.62 (s, 3H, Me), 3.95 (t, 1H, J 7.2 Hz, H3), 4.62 (d, 2H, J 6.8 Hz, H2), 6.59 (d, 1H, J 2.2 Hz, H8), 6.66 (d, 1H, J 2.2 Hz, H5), 7.07 (d, 2H, J 8.3 Hz, ArH), 7.28 (d, 2H, J 8.3 Hz, ArH).

### EXAMPLE 7

### Synthesis of 4′,7-Dihydroxy-5-methylisoflavanone

Imidazole (0.63 g) was added to a suspension of 4′,7-diacetoxy-5-methylisoflavan-4-one (0.50 g, 1.4 mmol) in absolute ethanol (20.0 ml) and the mixture was refluxed for 45 min under argon. The solution was concentrated under reduced pressure and distilled water (10 ml) was added to the residue. The mixture was left overnight in the fridge and the resulting precipitate was filtered, washed with water and dried to yield 4′,7-dihydroxy-5-methylisoflavan-4-one (0.25 g, 66%) as a white powder. ¹H NMR (d₆-acetone): δ 2.51 (s, 3H, Me), 3.76 (t, 1H, J 5.7 Hz, H3), 4.57 (d, 2H, J 7.1 Hz, H2), 6.26 (d, 1H, J 2.2 Hz, H8), 6.35 (d, 1H, J 2.2 Hz, H5), 6.78 (d, 2H, J 8.7 Hz, ArH), 7.11 (d, 2H, J 8.7 Hz, ArH).

Isolflavan-4-ols and Isoflav-3-enes

### EXAMPLE 8

### Synthesis of 4′-7-Diacetoxy-5-methylisoflavan-4-ol

4′-7-Diacetoxy-5-methylisoflavan-4-ol was prepared by the reduction of 4′-7-diacetoxy-5-methylisoflavone (0.25 g) with Adam's catalyst in ethyl acetate (30 ml) under a hydrogen atmosphere for 72 hours. The solution was filtered through a pad of Celite to yield predominantly cis-4′-7-diacetoxy-5-methylisoflavan-4-ol. ¹H NMR (CDCl₃): δ 2.26 (s, 3H, OCOCH₃), 2.30 (s, 3H, OCOCH₃), 2.62 (s, 3H, Me), 3.24 (dt, 1H, J 3.4 Hz, J 11.8 Hz, H3), 4.31 (ddd, 1H, J 1.4 Hz, 3.6 Hz, 10.5 Hz, H2); 4.57 (dd, 1H, J 10.5 Hz, 11.8 Hz, H2), 4.82 (bs, 1H, H4), 6.51 (d, 1H, J 2.1 Hz, H8), 6.59 (d, 1H, J 2.1 Hz, H6), 7.06 (d, 2H, J 8.6 Hz, ArH), 7.29 (d, 2H, J 8.6 Hz ArH).

### EXAMPLE 9

### Synthesis of 4′,7-Diacetoxy-5-methylisoflav-3-ene

4′,7-Diacetoxy-5-methylisoflav-3-ene was prepared by the dehydration of cis- and trans-4′-7-diacetoxy-5-methylisoflavan-4-ol (0.2 g) with phosphorus pentoxide (2.0 g) in dry dichloromethane (20 ml). The crude product was chromatographed on silica column using dichloromethane as the eluent. ¹H NMR (CDCl₃): δ 2.28 (s, 3H, OCOCH₃), 2.31 (s, 3H, OCOCH₃), 2.36 (s, 3H, Me), 5.08 (s, 2H, H2), 6.49 (d, 1H, J 2.0 Hz, H8), 6.52 (d, 1H, J 2.2 Hz, H5), 6.89 (s, 1H, H4), 7.14 (d, 2H, J 8.6 Hz, ArH), 7.44 (d, 2H, J 8.6 Hz, ArH).

### EXAMPLE 10

### Synthesis of 4′,7-Dihydroxy-5-methylisoflav-3-ene

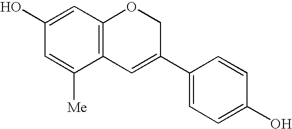

4′,7-Dihydroxy-5-methylisoflav-3-ene was prepared from 4′,7-diacetoxy-5-methylisoflav-3-ene by the removal of the acetoxy groups by hydrolysis under standard conditions.

### EXAMPLE 11

### Synthesis of 3′,5,7-Trihydroxyisoflavylium chloride

Phosphoryl chloride (1.75 ml) was added to a mixture of the monoaldehyde (0.95 g) and phloroglucinol dihydrate (1.6 g) in acetonitrile (10 ml). The mixture was stirred at 30° C. for 20 minutes and then at room temperature for 3 hours. The orange precipitate was filtered and washed with acetic acid to yield the isoflavylium salt.

### EXAMPLE 12

### Synthesis of Isoflav-3-ene-3′,5,7-triol

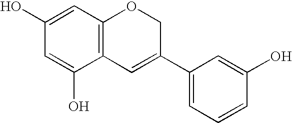

Isoflav-3-ene-3′,5,7-triol was prepared by the reduction of 3′,5,7-trihydroxyisoflavylium chloride (0.5 g) with sodium cyanoborohydride (0.33 g) in ethyl acetate (11 ml) and acetic acid (3 ml) and chromatographic separation of the resulting mixture of isoflav-3-ene and isoflav-2-ene mixture. ¹H NMR (d₆-acetone): δ 4.99 (s, 2H, H2), 5.92 (d, 1H, J 2.0 Hz, ArH), 6.04 (d, 1H, J 2.2 Hz, ArH), 6.78-7.18 (m, 5H, ArH).

### EXAMPLE 13

### Synthesis of 4′,7-Dihydroxy-8-methylisoflav-3-ene

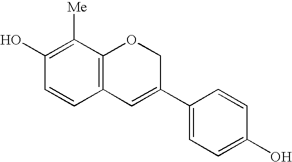

A mixture of 4′,7-dihydroxy-8-methylisoflavone (2.9 g, 10.8 mmol), acetic anhydride (18 ml) and pyridine (3 ml) was heated on an oil bath at 105-110° C. for 1 h. After cooling the mixture to room temperature, it was stirred for a further 30 min during which time the diacetate crystallised from the solution. The product was filtered, washed thoroughly with water and recrystallised from ethyl acetate to yield 4′,7-diacetoxy-8-methylisoflavone as colourless prisms (3.2 g, 84%). ¹H NMR (CDCl₃): δ 2.31 (s, 3H, CH₃), 2.32, 2.39 (each s, 3H, OCOCH₃), 7.13 (d, 1H, J 9.0 Hz, H6), 7.17 (d, 2H, J 8.7 Hz, ArH), 7.59 (d, 2H, J 8.7 Hz, ArH), 8.07 (s, 1H, H2), 8.19 (d, 1H, J 8.7 Hz, H5).

Palladium-on-charcoal (5%, 0.12 g) was added to a suspension of 4′,7-diacetoxy-8-methylisoflavone (1.0 g, 2.8 mmol) in methanol (200 ml) and the mixture was stirred at room temperature under a hydrogen atmosphere for 55 h. The catalyst was removed by filtration through Celite and the filtrate was evaporated in vacuo to yield 4′,7-diacetoxy-8-methylisoflavan-4-ol in quantitative yield, m.p. 135-37° C. A nuclear magnetic resonance spectrum revealed the product to be a clean 1:1 mixture of cis- and trans-4′,7-diacetoxy-8-methylisoflavan-4-ol. Mass spectrum: 356 (M, 53%); 254 (86); 253 (100); 240 (80); 196 (37).

For trans-4′,7-diacetoxy-8-methylisoflavan-4-ol; ¹H NMR (CDCl₃): δ 2.02 (s, 3H, CH₃), 2.30, 2.31 (each s, 3H, OCOCH₃), 3.15 (ddd, 1H, J 3.8 Hz, 8.6 Hz, 11.7, H3), 4.27 (dd, 1H, J 9.4 Hz, 11.3 Hz, H2); 4.39 (m, 1H, H2), 4.92 (d, 1H, J 7.5 Hz, H4), 6.64 (d, 1H, J 8.0 Hz, H6), 7.06-7.32 (m, ArH).

For cis-4′,7-diacetoxy-8-methylisoflavan-4-ol; ¹H NMR (CDCl₃): δ 2.02 (s, 3H, CH₃), 2.31, 2.32 (each s, 3H, OCOCH₃), 3.28 (dt, 1H, J 3.4 Hz, J 11.7 Hz, H3), 4.40 (m, 1H, H2); 4.58 (dd, 1H, J 10.1 Hz, 11.7 Hz, H2), 4.78 (bs, 1H, H4), 6.67 (d, 1H, J 8.0 Hz, H6), 7.06-7.32 (m, ArH).

Phosphorus pentoxide (3.0 g) was added with stirring to a solution of cis- and trans-4′,7-diacetoxy-8-methylisoflavan-4-ol (0.55 g, 1.5 mmol) in dry dichloromethane (25 ml). The mixture was stirred at room temperature for 2 h and filtered through a pad of Celite. The resulting solution was concentrated and chromatographed on silica gel to yield 4′,7-diacetoxy-8-methylisoflav-3-ene (0.25 g, 48%). m.p. 140° C. ¹H NMR (CDCl₃): δ 2.04 (s, 3H, CH₃), 2.31, 2.32 (each s, 3H, OCOCH₃), 5.16 (s, 2H, H2), 6.61 (d, 1H, J 8.3 Hz, H6), 6.75 (bs, 1H, H4), 6.94 (d, 1H, J 8.3 Hz, H5), 7.13 (d, 2H, J 8.7 Hz, ArH), 7.45 (d, 2H, J 8.7 Hz, ArH). Mass spectrum: m/z 339 (M+1, 6%); 338 (M, 26); 296 (48); 254 (90); 253 (100).

Imidazole (0.6 g) was added to a suspension of 4′,7-diacetoxy-8-methylisoflav-3-ene (0.25 g, 0.7 mmol) in absolute ethanol (5.0 ml) and the mixture was refluxed for 45 min under argon. The solution was concentrated under reduced pressure and the product was precipitated by addition of distilled water (10 ml). The mixture was left overnight in the fridge and filtered to yield isoflav-3-ene. The crude product was recrystallised from methanol/benzene to yield 8-methylisoflav-3-ene-4′,7-diol (0.13 g, 68%). m.p. 190-93° C. ¹H NMR (CDCl₃+d₆-DMSO): δ 1.94 (s, 3H, CH₃), 4.98 (s, 2H, H2), 6.32 (d, 1H, J 7.9 Hz, H6), 6.58 (bs, 1H, H4), 6.67 (bd, 1H, H5), 6.72 (d, 2H, J 8.7 Hz, ArH), 7.21 (bd, 2H, ArH). Mass spectrum: m/z 255 (M+1, 16%); 254 (M, 79); 253 (100); 161 (32).

### EXAMPLE 14

### Synthesis of 3′,7-Dihydroxy-8-methylisoflav-3-ene

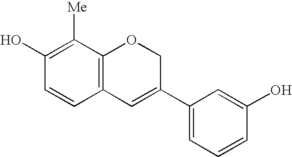

3′,7-Diacetoxy-8-methylisoflavone was prepared from 3′,7-dihydroxy-8-methylisoflavone (1.3 g, 4.8 mmol), acetic anhydride (8 ml) and pyridine (1.5 ml) as described for 4′,7-diacetoxy-8-methylisoflavone. Yield: (1.2 g, 70%) m.p. 112° C. ¹H NMR (CDCl₃): δ2.31 (s, 3H, CH₃), 2.32, 2.39 (each s, 3H, OCOCH₃), 7.13 (m, 2H, ArH), 7.37-7.45 (m, 3H, ArH), 8.1 (s, 1H, H2), 8.18 (d, 1H, J 8.7 Hz, H5). Mass spectrum: m/z 352 (M, 6%); 310 (35); 268 (100); 267 (60).

3′,7-Diacetoxy-8-methylisoflavan-4-ol was prepared from 3′,7-diacetoxy-8-methylisoflavone (0.25 g, 0.7 mmol) in methanol (50 ml) using palladium-on-charcoal (5%, 0.06 g) by the method described above.

For trans-3′,7-diacetoxy-8-methylisoflavan-4-ol; ¹H NMR (CDCl₃): δ 2.03 (s, 3H, CH₃), 2.30, 2.32 (each s, 3H, OCOCH₃), 3.18 (ddd, 1H, J 3.8 Hz, 8.3 Hz, 12.1 Hz, H3), 4.28 (dd, 1H, J 9.0 Hz, 10.9 Hz, H2); 4.39 (m, 1H, H2), 4.94 (d, 1H, J 8.7 Hz, H4), 6.65 (d, 1H, J 7.9 Hz, H6), 6.98-7.39 (m, ArH).

For cis-3′,7-diacetoxy-8-methylisoflavan-4-ol; ¹H NMR (CDCl₃): δ 2.05 (s, 3H, CH₃), 2.30, 2.32 (each s, 3H, OCOCH₃), 3.32 (dt, 1H, J 3.4 Hz, J 12.0 Hz, H3), 4.39 (m, 1H, H2); 4.59 (dd, 1H, J 10.5 Hz, 11.7 Hz, H2), 4.80 (bs, 1H, H4), 6.68 (d, 1H, J 8.3 Hz, H6), 6.98-7.39 (m, ArH).

3′,7-Diacetoxy-8-methylisoflav-3-ene was prepared from cis- and trans-3′,7-diacetoxy-8-methylisoflavan-4-ol (0.25 g, 0.7 mmol) in dry dichloromethane (20 ml) using phosphorus pentoxide (2.0 g). Yield: (0.13 g, 54%) m.p. 116° C. ¹H NMR (CDCl₃): δ 2.04 (s, 3H, CH₃), 2.31, 2.32 (each s, 3H, OCOCH₃), 5.16 (s, 2H, H2), 6.61 (d, 1H, J 8.3 Hz, H6), 6.79 (bs, 1H, H4), 6.92 (d, 1H, J 8.3 Hz, ArH), 7.05 (dd, 1H, J 2.0 Hz, 8.0 Hz, ArH), 7.15 (s, 1H, ArH), 7.26 (d, 1H, J 8.0 Hz, ArH), 7.37 (t, 1H, J 8.0 Hz, ArH). Mass spectrum: m/z 339 (M+1, 15%); 338 (M, 22); 296 (54); 254 (30).

8-Methylisoflav-3-ene-3′,7-diol was prepared from 3′,7-diacetoxy-8-methylisoflav-3-ene (0.12 g, 0.4 mmol) and imidazole (0.3 g) in ethanol (2.5 ml) as described for 8-methylisoflav-3-ene-4′,7-diol. Yield: (0.07 g, 77%) m.p. 130° C. ¹H NMR (CDCl₃+d₆-DMSO): δ 1.95 (s, 3H, CH₃), 4.98 (s, 2H, H2), 6.34 (d, 1H, J 8.0 Hz, H6), 6.61-6.94 (m, 5H, ArH), 7.08 (bt, 1H, ArH). Mass spectrum: m/z 254 (M, 100%); 253 (96); 161 (45).

### EXAMPLE 15

### Synthesis of 4′,7-Dihydroxy-3′-methoxy-8-methylisoflav-3-ene

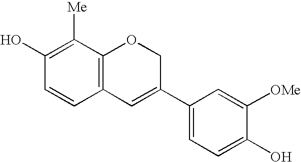

4′,7-Diacetoxy-3′-methoxy-8-methylisoflavone was prepared from 4′,7-dihydroxy-3′-methoxy-8-methylisoflavone (0.42 g, 1.4 mmol), acetic anhydride (2.6 ml) and pyridine (0.5 ml) as described for 4′,7-diacetoxy-8-methylisoflavone. Yield: (0.4 g, 74%) m.p. 209° C. ¹H NMR (CDCl₃): δ 2.22 (s, 3H, CH₃), 2.32, 2.39 (each s, 3H, OCOCH₃), 3.89 (s, 3H, OMe), 7.07-7.11 (m, 2H, ArH), 7.13 (d, 1H, J 8.6 Hz, H6), 7.32 (d, 1H, J 1.5 Hz, ArH), 8.09 (s, 1H, H2), 8.18 (d, 1H, J 8.7 Hz, H5).

4′,7-Diacetoxy-3′-methoxy-8-methylisoflavan-4-ol was prepared from 4′,7-diacetoxy-3′-methoxy-8-methylisoflavone (0.25 g, 0.7 mmol) in methanol (50 ml) using palladium-on-charcoal (5%, 0.07 g) by the method described above.

For trans-4′,7-diacetoxy-3′-methoxy-8-methylisoflavan-4-ol; ¹H NMR (CDCl₃): δ 2.05 (s, 3H, CH₃), 2.30, 2.32 (each s, 3H, OCOCH₃), 3.18 (ddd, 1H, J 3.8 Hz, 8.3 Hz, 11.4 Hz, H3), 3.79 (s, 3H, OMe), 4.28 (dd, 1H, J 9.0 Hz, 11.3 Hz, H2); 4.41 (m, 1H, H2), 4.93 (d, 1H, J 7.9 Hz, H4), 6.64 (d, 1H, J 7.9 Hz, H6), 6.75-6.92 (m, ArH), 7.00 (d, 1H, J 7.9 Hz, ArH), 7.16 (d, 1H, J 8.3 Hz, ArH).

For cis-3′,7-diacetoxy-8-methylisoflavan-4-ol; ¹H NMR (CDCl₃): δ 2.05 (s, 3H, CH₃), 2.30, 2.32 (each s, 3H, OCOCH₃), 3.29 (dt, 1H, J 3.4 Hz, J 11.7 Hz, H3), 4.40 (m, 1H, H2); 4.59 (t, 1H, J 10.5 Hz, H2), 4.81 (bs, 1H, H4), 6.67 (d, 1H, J 7.9 Hz, H6), 6.75-6.92 (m, ArH), 7.03 (d, 1H, J 8.3 Hz, ArH), 7.33 (d, 1H, J 8.3 Hz, ArH).

4′,7-Diacetoxy-3′-methoxy-8-methylisoflav-3-ene was prepared from cis- and trans-4′.7-diacetoxy-3′-methoxy-8-methylisoflavan-4-ol (0.25 g, 0.6 mmol) in dry dichloromethane (25 ml) using phosphorus pentoxide (2.0 g). Yield: (0.14 g, 58%) m.p. 123° C. ¹H NMR (CDCl₃): δ 2.05 (s, 3H, CH₃), 2.31, 2.32 (each s, 3H, OCOCH₃), 3.88 (s, 3H, OMe), 5.16 (s, 2H, H2), 6.61 (d, 1H, J 8.3 Hz, H6), 6.73 (bs, 1H, H4), 6.94 (d, 1H, J 8.3 Hz, H5), 6.97 (dd, 1H, J 1.9 Hz, 8.3 Hz, ArH), 7.03 (d, 1H, J 1.9 Hz, ArH), 7.05 (d, 1H, J 7.9 Hz, ArH).

3′-Methoxy-8-methylisoflav-3-ene-4′,7-diol was prepared from 4′,7-diacetoxy-3′-methoxy-8-methylisoflav-3-ene (0.21 g, 0.6 mmol) and imidazole (0.52 g) in ethanol (4 ml) as described for 8-methylisoflav-3-ene-4′,7-diol. Yield: (0.1 g, 63%). ¹H NMR (CDCl₃): δ 2.14 (s, 3H, CH₃), 3.94 (s, 3H, OMe), 5.11 (s, 2H, H2), 6.42 (d, 1H, J 8.3 Hz, H6), 6.64 (bs, 1H, ArH), 6.80 (d, 1H, J 7.9 Hz, ArH), 6.94 (m, 2H, ArH), 7.12 (m, 1H, ArH), 7.26, 7.70 (each bs, 1H, OH).

### EXAMPLE 16

### Synthesis of 7-Hydroxy-3′-methoxyisoflav-3-ene

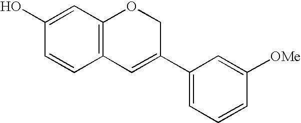

7-Acetoxy-3′-methoxyisoflavone was prepared from 7-hydroxy-3′-methoxyisoflavone (1.7 g, 6.3 mmol), acetic anhydride (6 ml) and pyridine (1.0 ml) as described for 4′,7-diacetoxydaidzein. Yield: (1.6 g, 81%) m.p. 118° C. ¹H NMR (CDCl₃): δ 2.36 (s, 3H, OCOCH₃), 3.85 (s, 3H, OMe), 6.95 (dd, 1H, J 2.0 Hz 8.3 Hz, H6), 6.70-7.40 (m, 5H, ArH), 8.01 (s, 1H, H2), 8.32 (d, 1H, J 8.7 Hz, H5).

Cis- and trans-7-acetoxy-3′-methoxyisoflavan-4-ol was prepared from 7-acetoxy-3′-methoxyisoflavone (0.5 g, 1.6 mmol) and palladium-on-charcoal (5%, 0.12 g) in methanol (100 ml) by the method described above.

For trans-7-acetoxy-3′-methoxyisoflavan-4-ol; ¹H NMR (CDCl₃): δ 2.28 (s, 3H, OCOCH₃), 3.15 (ddd, 1H, J 3.8 Hz, 8.3 Hz, 12.0 Hz, H3), 3.80 (s, 3H, OMe), 4.26 (dd, 1H, J 9.4 Hz, 11.3 Hz, H2); 4.32 (m, 1H, H2), 4.95 (d, 1H, J 7.9 Hz, H4), 6.60-6.93 (m, ArH), 7.23-7.33 (m, ArH), 7.49 (d, J 8.7 Hz, ArH).

For cis-7-acetoxy-3′-methoxyisoflavan-4-ol; ¹H NMR (CDCl₃): δ 2.28 (s, 3H, OCOCH₃), 3.30 (dt, 1H, J 3.3 Hz, J 11.7 Hz, H3), 4.31 (m, 1H, H2); 4.58 (dd, 1H, J 10.5 Hz, 11.7 Hz, H2), 4.81 (bs, 1H, H4), 6.60-6.93 (m, ArH), 7.23-7.33 (m, ArH), 7.49 (d, J 8.7 Hz, ArH).

7-Acetoxy-3′-methoxyisoflav-3-ene was prepared from cis- and trans-7-acetoxy-3′-methoxyisoflavan-4-ol (0.25 g, 0.8 mmol) in dry dichloromethane (20 ml) using phosphorus pentoxide (2.0 g). Yield: (0.15 g, 63%). ¹H NMR (CDCl₃): δ 2.28 (s, 3H, OCOCH₃), 3.85 (s, 3H, OMe), 5.15 (s, 2H, H2), 6.60-6.67 (m, 2H, ArH), 6.78 (bs, 1H, H4), 6.84-7.06 (m, 4H, ArH), 7.35 (t, 1H, J 8.6 Hz, ArH).

3′-Methoxylsoflav-3-ene-7-ol was prepared from 7-acetoxy-3′-methoxyisoflav-3-ene (0.1 g, 0.3 mmol) and imidazole (0.15 g) in ethanol (2.0 ml) as described for isoflav-3-ene-4′,7-diol. Yield: (0.06 g, 70%) m.p. 75° C. ¹H NMR (CDCl₃): δ 3.84 (s, 3H, OMe), 5.12 (s, 2H, H2), 6.38 (d, 1H, J 2.0 Hz, H8), 6.40 (dd, 1H, J 2.0 Hz, 8.3 Hz, H6), 6.76 (bs, 1H, H4), 6.84 (dd, 1H, J 1.9 Hz, 8.3 Hz, ArH), 6.95 (m, 3H, ArH), 7.29 (t, 1H, J 8.3 Hz, ArH).

### EXAMPLE 17

### Synthesis of 7-Hydroxy-8-methylisoflav-3-ene

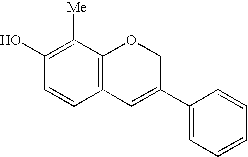

2-Methyl-resorcinol and phenyl acetic acid were combined in a round bottom flask and flushed with nitrogen according to the general procedure below. Boron trifluoride diethyl etherate was added to the solids in the flask and the mixture was stirred under nitrogen with heating to 110° C., forming a brown mass. The mixture was then cooled to room temperature for 2 hours and the resulting precipitate was collected and washed with an excess of water to afford 4-hydroxy-3-methyldeoxybenzoin.

4-Hydroxy-3-methyldeoxybenzoin (92 g) dissolved in N,N-DMF (140 mL) was placed under a nitrogen atmosphere. Distilled boron trifluoride diethyl etherate was added over 40 min to the stirred solution at room temperature. A solution of methanesulfonyl chloride in N,N-DMF was added at 55° C. over 20 min. During the addition of methanesulfonyl chloride solution, the reaction mixture changed to a yellow colour. The reaction was heated to reflux for 80 min and was then left to cool to room temperature. The dark brown solution was poured into cold, vigourously stirred water (in portions). Overnight (with continued stirring) the yellow solid precipitated out. The solid was washed with water and collected by filtration. The solid was dried to yield 7-hydroxy-8-methylisoflavone as a yellow solid (94 g, 99%).

The 7-hydroxy-8-methylisoflavone and acetic acid were combined in a round bottom flask and pyridine was added drop wise. The mixture was heated to reflux for 2 h before being cooled to room temperature. Orange crystals formed on cooling and were collected by suction filtration and washed with water to afford 7-acetoxy-8-methylisoflavone.

Palladium on alumina (10%) was added to a solution of 7-acetoxy-8-methylisoflavone in ethanol and the mixture was stirred at room temperature under a hydrogen atmosphere for 2 h. The catalyst was removed by filtering through Celite and the filtrate was evaporated in vacuo to afford a cream coloured mixture of cis- and trans-7-acytoxy-8-methylisoflavan-4-ol.

7-Acetoxy-8-methylisoflav-3-ene was prepared by dehydration of cis- and trans-7-acytoxy-8-methylisoflavan-4-ol by phosphorus pentoxide in dry DCM. The crude product was separated on a silica column with DCM and ethyl acetate before evaporating in vacuo.

The mono-acetoxy compound from above was weighed into a round bottom flask and dissolved in methanol. Potassium hydroxide solution was added dropwise to the stirred solution. The reaction was complete after 15 mins and was neutralised with acetic acid solution. The reaction mixture was poured into ice cold water producing a precipitate. The precipitate was filtered through a 0.45 μm filter to afford the title compound, 7-hydroxy-8-methylisoflav-3-ene.

¹H NMR (400 MHz, d₆-DMSO): δ 1.98 (s, 3H, CH₃), 5.11 (s, 2H, H2), 6.40 (d, 1H, J=8.0 Hz, H6), 6.83 (d, 1H, J=8.1 Hz, H5), 6.94 (s, 1H, H4), 7.26 (dd, 1H, J=7.3 Hz, H4′), 7.38 (dd, 2H, J=7.7 Hz, H3′ H5′), 7.49 (d, 2H, J=8.0 Hz, H2′ H6′), 9.51 (br s, 1H, OH).

### EXAMPLE 18

### Synthesis of 7-Hydroxy-3′,4′-dimethoxyisoflav-3-ene

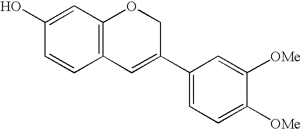

Resorcinol (1.5 g) and 3,4-methoxyphenyl acetic acid (2 g) were combined in a round bottom flask and flushed with nitrogen. Boron trifluoride diethyl etherate (5.5 mL) was added to the solids in the flask and the mixture was stirred under nitrogen with heating to 110° C., forming an orange mass. The mixture was then cooled to room temperature for 2 hours.

N,N-DMF (5 mL) was added to the flask over 20 minutes to dissolve the solid mass. Distilled boron trifluoride diethyl etherate (4 mL) was added over 40 min to the stirred solution at room temperature. The mixture was heated to 50° C. wherein a solution of methanesulfonyl chloride (2 mL) in N,N-DMF (6 mL) was added over 20 min. The mixture was slowly heated to 110° C. for 2 h before allowing to cool to room temperature. The dark brown solution was poured into cold, vigourously stirred water (300 mL). Overnight (with continued stirring) the orange solid precipitated out. The solid was washed with water and collected by suction filtration to afford 3′,4′-dimethoxy-7-hydroxyisoflavone (2.7 g, 80%).

The 3′,4′-dimethoxy-7-hydroxyisoflavone (2.7 g) and acetic acid (15 mL) were combined in a round bottom flask and pyridine (2 mL) was added dropwise. The mixture was heated to reflux for 2 h before being cooled to room temperature. The solution was poured into cold water (600 mL) forming a yellow solid. The solid was collected by suction filtration, washed with water and recrystallised from ethyl acetate to afford white 7-acytoxy-3′,4′-dimethoxyisoflavone (1 g).

Palladium on alumina (10%, 0.05 g) was added to a solution of 7-acytoxy-3′,4′-dimethoxyisoflavone (0.5 g) in ethanol (30 mL) and the mixture was stirred at room temperature under a hydrogen atmosphere for 48 h. The catalyst was removed by filtering through Celite and the filtrate was evaporated in vacuo to afford a mixture of cis and trans 7-acytoxy-3′,4′-dimethoxy isoflavan-4-ol.

7-Acetoxy-3′,4′-dimethoxyisoflav-3-ene was prepared by dehydration of cis- and trans-7-acytoxy-3′,4′-dimethoxy isoflavan-4-ol (0.4 g) by phosphorus pentoxide (4.5 g) in dry DCM (20 mL). The crude product was chromatograped on a silica column with DCM and ethyl acetate before evaporating in vacuo.

The mono-acetoxy compound (63 mg) and methanol (5 mL) were combined in a round bottom flask and potassium hydroxide (1 mL, 1 M) was added drop wise causing the clear white solution to become a clear yellow solution. The solution was neutralised with acetic acid and reduced under vacuo before being poured into chilled distilled water stirring vigorously. The solution was allowed to stir overnight at 4° C., producing a white precipitate which was collected by suction filtration to afford the title compound, 7-hydroxy-3′,4′-dimethoxyisoflav-3-ene (33 mg).

¹H n.m.r. (400 MHz, d₆-DMSO): δ 3.76 (3H, s, —OCH₃), 3.81 (3H, s, —OCH₃), 5.05 (2H, s, H2), 6.25 (1H, d, J=2.3, H8), 6.34 (1H, dd, J=2.3, 8.2, H6), 6.88 (1H, s, H4), 6.92-7.00 (3H, m, H2′ H5′ H6′), 7.12 (1H, d, J=1.8, H5), 9.57 (1H, s, —OH).

### EXAMPLE 19

### Synthesis of 7-Hydroxy-3′,4′-dimethoxy-8-methylisoflav-3-ene

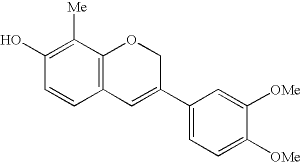

2-Methyl-resorcinol (62 g) and 3,4-methoxyphenyl acetic acid (92 g) were combined in a round bottom flask and flushed with nitrogen. Boron trifluoride diethyl etherate (350 mL) was added to the solids in the flask and the mixture was stirred under nitrogen with heating to 110° C., forming a brown mass. The mixture was then cooled to room temperature for 2 hours and the resulting precipitate was collected and washed with an excess of water to afford 3′,4′-dimethoxy-4-hydroxy-3-methyldeoxybenzoin (93 g, 65%).

3′,4′-Dimethoxy-4-hydroxy-3-methyldeoxybenzoin (92 g) dissolved in N,N-DMF (140 mL) was placed under a nitrogen atmosphere. Distilled boron trifluoride diethyl etherate was added (140 mL) over 40 min to the stirred solution at room temperature. A solution of methanesulfonyl chloride (75 mL) in N,N-DMF (190 mL) was added at 55° C. over 20 min. During the addition of methanesulfonyl chloride solution, the reaction mixture changed to a yellow colour. The reaction was heated to reflux for 80 min and was then left to cool to room temperature. The dark brown solution was poured into cold, vigourously stirred water (3×1250 mL portions). Overnight (with continued stirring) the yellow solid precipitated out. The solid was washed with water and collected by filtration. The solid was dried to yield 3′,4′-dimethoxy-7-hydroxy-8-methylisoflavone as a yellow solid (94 g, 99%).

The 3′,4′-dimethoxy-7-hydroxy-8-methylisoflavone (22 g) and acetic acid (138 mL) were combined in a round bottom flask and pyridine (8 mL) was added drop wise. The mixture was heated to reflux for 2 h before being cooled to room temperature. Orange crystals formed on cooling and were collected by suction filtration and washed with water to afford 7-acetoxy-3′,4′-dimethoxy-8-methylisoflavone (7 g).

Palladium on alumina (10%, 1.5 g) was added to a solution of 7-acetoxy-3′,4′-dimethoxy-8-methylisoflavone (4 g) in ethanol (600 mL) and the mixture was stirred at room temperature under a hydrogen atmosphere for 2 h. The catalyst was removed by filtering through Celite and the filtrate was evaporated in vacuo to afford a cream coloured mixture of cis- and trans-7-acytoxy-3′,4′-dimethoxy-8-methylisoflavan-4-ol.

7-Acetoxy-3′,4′-dimethoxy-8-methylisoflav-3-ene was prepared by dehydration of cis- and trans-7-acytoxy-3′,4′-dimethoxy-8-methylisoflavan-4-ol (0.4 g) by phosphorus pentoxide (4.5 g) in dry DCM (20 mL). The crude product was separated on a silica column with DCM and ethyl acetate before evaporating in vacuo.

The mono-acetoxy compound from above (187 mg) was weighed into a round bottom flask and dissolved in methanol (20 ml). Potassium hydroxide solution (2 mL) was added dropwise to the stirred solution. The reaction was complete after 15 mins and was neutralised with acetic acid solution (2 mL). The reaction mixture was poured into ice cold water (150 ml) producing a precipitate. The precipitate was filtered through a 0.45 μm filter to afford the title compound, 7-hydroxy-3′,4′-dimethoxy-8-methylisoflav-3-ene (111 mg, >95% purity).

¹H n.m.r. (400 MHz, d₆-DMSO): δ 1.97 (3H, s, OCH₃), 3.76 (3H, s, OCH₃), 3.81 (3H, s, OCH₃), 5.07 (2H, s, H2), 6.39 (11H, d, J=8.4, H6), 6.80 (1H, d, J=8.1, H5), 6.86 (1H, s, H4), 6.92 (1H, d, J=8.4 Hz, H5′), 6.97 (1H, dd, J=8.4, 2.0 Hz, H6′), 7.12 (1H, d, J=1.9 Hz, H2′), 9.50 (1H, br s, OH).

### EXAMPLE 20

### Synthesis of 4′,7-Dihydroxy-8-bromoisoflav-3-ene

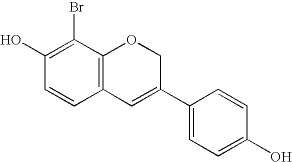

2-Bromoresorcinol (8.5 g) and 4-hydroxyphenylacetic acid (8 g) were combined in a round bottom flask and flushed with nitrogen. Boron trifluoride diethyl etherate (50 mL) was added to the solids in the flask and the mixture was stirred under nitrogen with heating to 110° C., for 100 min. The mixture was then cooled to room temperature for 2 hours and the resulting yellow precipitate was collected and washed with an excess of water to afford 3′,4-dihydroxy-3-bromodeoxybenzoin (7.7 g, 52%). 3′,4-Dihydroxy-3-bromodeoxybenzoin (7.7 g) dissolved in N,N-DMF (90 mL) was placed under a nitrogen atmosphere. Distilled boron trifluoride diethyl etherate was added (27 mL) over 40 min to the stirred solution at room temperature. A solution of methanesulfonyl chloride (15 mL) was added at 55° C. over 20 min. The reaction was heated to 110° C. under reflux for 120 min and was then left to cool to room temperature, whereby 2 M HCl (330 mL) was added to the crude reacted solution. A yellow precipitate was produced and isolated via suction filtration and dried in vacuo to afford 8-bromo-4′,7-dihydroxyisoflavone (4 g, 50.4%).

To a solution of 8-bromo-4′,7-dihydroxyisoflavone (3.9 g) in acetic anhydride (30 mL), was added pyridine (2 mL). The reaction mixture was heated at 110° C. for 1 hour then cooled. The resulting yellow precipitate was collected via vacuum filtration to afford 4′,7-diacetoxy-8-bromoisoflavone (2.738 g, 55%).

Cerium chloride heptahydrate (120 mg) was added to 4′,7-diacetoxy-8-bromoisoflavone (520 mg) dissolved in methanol (500 mL) and stirred under N₂ at 0° C. Sodium borohydride (33 mg) was added over four lots and stirred at 0° C. to give a yellow solution. The solution was reduced in vacuo and extracted with DCM and water. Organic DCM layer afforded a white solid, 4′,7-diacytoxy-8-bromoisoflavan-4-ol (181.2 mg, 35%).

4′,7-Diacytoxy-8-bromoisoflavan-4-ol (1.1 g) was dissolved in DCM (35 mL) in a round bottom flask fixed with a silica drying tube. Phosphorous pentoxide (2.2 g) was then added and allowed to stir at room temperature for 35 minutes. The reaction mixture was run through a silica plug with ethyl acetate (600 mL) and was reduced in vacuo to afford 4′, 7-diacetoxy-8-bromoisoflav-3-ene (1.05 g, 98%) as a white solid.

To a solution of 4′,7-diacetoxy-8-bromoisoflav-3-ene (802 mg) in ethanol (40 mL) was added potassium hydroxide (5 mL), drop wise whilst stirring. The solution was neutralised with acetic acid after 2 hours before being reduced in vacuo to ˜15 mL and poured into chilled water (200 mL) and stirred overnight. Suction filtration afforded the title compound, 4′,7-dihydroxy-8-bromoisoflav-3-ene (628.4 mg, 100%) as a salmon pink solid.

¹H n.m.r. (400 MHz, d₃₆-DMSO): δ 5.13 (1H, s, H2), 6.47 (1H, d, J=8.2, H6), 6.78 (2H, d, J=8.7, H3′ H5′), 6.79 (1H, s, H4), 6.95 (1H, d, J=8.2, H5), 7.36 (2H, d, J=8.7, H2′H6′), 9.62 (1H, s, OH), 10.27 (1H, s, OH).

### EXAMPLE 21

### Synthesis of 4′-Bromo-7-hydroxyisoflav-3-ene

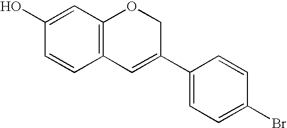

Resorcinol and 4-bromophenyl acetic acid were combined in a round bottom flask and flushed with nitrogen. Boron trifluoride diethyl etherate was added to the solids in the flask and the mixture was stirred under nitrogen with heating to 110° C., forming an orange mass. The mixture was then cooled to room temperature.

N,N-DMF was added to the flask over 20 minutes to dissolve the solid mass. Distilled boron trifluoride diethyl etherate was added over 40 min to the stirred solution at room temperature. The mixture was heated to 50° C. wherein a solution of methanesulfonyl chloride in N,N-DMF was added over 20 min. The mixture was slowly heated to 110° C. for 2 h before allowing to cool to room temperature. The dark brown solution was poured into cold, vigourously stirred water. Overnight (with continued stirring) the solid precipitated out. The solid was washed with water and collected by suction filtration to afford 7-hydroxy-4′-bromoisoflavone.

The 7-hydroxy-4′-bromoisoflavone and acetic acid were combined in a round bottom flask and pyridine (2 mL) was added drop wise. The mixture was heated to reflux for 2 h before being cooled to room temperature. The solution was poured into cold water forming a yellow solid. The solid was collected by suction filtration, washed with water and recrystallised from ethyl acetate to afford white 7-acytoxy-4′-bromoisoflavone.

Platinum oxide (20%, 4.16 g) was added to a solution of 7-acytoxy-4′-bromoisoflavone (1.7 g) in dry ethyl acetate (150 mL) and the mixture was stirred at room temperature under a hydrogen atmosphere for 2 h. The catalyst was removed by filtering through Celite and the filtrate was evaporated in vacuo to afford a mixture of cis- and trans-7-acytoxy-4′-bromoisoflavan-4-one (1.5 g).

Cerium chloride heptahydrate (1.55 g) was added to 7-acytoxy-4′-bromoisoflavan-4-one (1.5 g) dissolved in methanol (100 mL) and stirred under N₂ at 0° C. Sodium borohydride (110 mg) was added over four lots and stirred at 0° C., before being quenched with ammonium chloride and extracted with ethyl acetate to afford a white solid, 7-acytoxy-4′-bromoisoflavan-4-ol (710 mg, 40%).

7-Acytoxy-4′-bromoisoflavan-4-ol (540 mg) was dissolved in DCM (20 mL) in a round bottom flask fixed with a silica drying tube. Phosphorous pentoxide (1.7 g) was then added and allowed to stir at room temperature for 35 minutes. The reaction mixture was run through a silica plug with ethyl acetate (400 mL) and was reduced in vacuo to afford 7-acetoxy-4′-bromoisoflav-3-ene (600 mg) as a white solid.

7-Acetoxy-4′-bromoisoflav-3-ene (600 mg) and imidazole (630 mg) were combined in a round bottom flask and dissolved in ethanol (35 mL). The mixture was refluxed under nitrogen for 5 hours, then cooled to room temperature. The solution was poured into cold water forming an off white precipitate. The solid was collected by suction filtration to afford the title compound, 7-hydroxy-4′-bromoisoflav-3-ene (90%).

¹H NMR (400 MHz, d₆-DMSO): δ 5.11 (s, 2H, H2), 6.25 (d, 1H, J=2.2 Hz, H8), 6.35 (dd, J=2.3, 8.2 Hz, 1H, H6), 7.00 (d, 1H, J=8.3 Hz, H5), 7.02 (d, 1H, J=8.7 Hz, H4), 7.45 (d, 2H, J 8.7 Hz, H2′H6′), 7.55 (d, 2H, J=8.0 Hz, H3′H5′), 9.67 (1H, br s, OH).

### Isoflavans

### EXAMPLE 22

### Synthesis of Isoflavan-5,7-diol

Isoflavan-5,7-diol was prepared by the reduction of a suspension of 5,7-dihydroxyisoflavylium chloride (0.5 g) with Palladium-on-charcoal (5%, 0.1 g) in acetic acid (15 ml) containing ethyl acetate (2.5 ml) under a hydrogen atmosphere. The crude product was recrystallised from 1,2-dichloromethane to give the isoflavan as colourless needles, m.p. 76-78° C. (lit m.p. 77-79° C.).

### EXAMPLE 23

### Synthesis of 4′,5,7-Triacetoxyisoflavan

4′,5,7-Triacetoxyisoflavan was prepared by the reduction of a suspension of 4′,5,7-trihydroxyisoflavylium chloride (0.31 g) with platinum oxide (0.04 g) in a mixture of acetic anhydride (2.0 ml) and ethyl acetate (10 ml) under a hydrogen atmosphere. After the removal of catalyst the crude product was refluxed with pyridine (0.5 ml) and the resulting triacetate was isolated by evaporation of the solvent and crystallisation of the residue. M.p. 126-28° C.

### EXAMPLE 24

### Synthesis of Isoflavan-4′,5,7-triol

Isoflavan-4′,5,7-triol was prepared from 4′,5,7-triacetoxyisoflavan by the removal of the acetyl groups by hydrolysis. M.p. 206-8° C.

### EXAMPLE 25

### In Vitro Activity

### 1. Estrogen Receptor Binding Activity

The binding affinity of various compounds of the invention for both subtypes of the estrogen receptor was determined with the “Estrogen Receptor Alpha or Beta Competitor Assay Core HTS Kit” supplied by Panvera Corporation (Product No. P2614/2615). 6-Chloro-4′,7-dihydroxyisoflavan-4-one showed good competitive binding to the estrogen receptor with the following results:
ER alpha receptor=37.82 uM
ER beta receptor=32.14 uM

### 2.1 Anti-Inflammatory Effects

### 2.1.1 Effect on NFκB Production

Nuclear factor-kappa B (NFκB) is a ubiquitous transcription factor that, by regulating the expression of multiple inflammatory and immune genes, plays a critical role in chronic inflammatory diseases. Consequently, its inhibition by anti-oxidative or anti-inflammatory agents is considered to be an anti-inflammatory strategy, and has become a target for novel therapeutics.

The influence of NFκB is particularly important in atherosclerosis. The NFκB regulatory pathway is oxidant-sensitive and is central to the transcription of several atherosclerosis-related genes eg leukocyte adhesion molecules and chemoattractant cytokines. The activated form of NFκB is present in atherosclerotic plaques, and the inhibition of NFκB may suppress endothelial activation and induce VSMC apoptosis in atherosclerotic lesions.

NFκB is also involved in the metabolic syndrome and insulin resistance. Visceral adipose tissue products, such as free fatty acids and their metabolites are thought to activate NFκB, and inhibit insulin signalling. Increased adipose tissue mass contributes to augmented secretion of proinflammatory adipokines, particularly TNFα, which activates NFκB. Elevated free fatty acid, glucose and insulin levels enhance this NFκB activation and further downstream modulate specific clinical manifestations of metabolic syndrome.

NFκB transcription factors are over expressed in rheumatoid arthritis (RA) patients. NFκB and its receptor activator, RANK are key regulators of bone remodelling and the T cell/dendritic cell regulation that occurs in the bone degeneration of arthritis.

It has been hypothesised that in psoriasis, defects in the regulation of NFκB cause a reduction in the control of keratinocyte growth and differentiation when the cells are subjected to physico-chemical and immunological stress.

Activated NFκB (NFκB p50) is widely expressed in the subretinal membranes of patients with AMD compared to those from healthy eyes. Diabetes is considered to increase the risk of KCS. In a rat model of diabetes, the expression of NFκB in lacrimal glands in diabetic rats suggests that these it is involved in the signalling and in subsequent inflammatory alterations related to dry eye in diabetes mellitus.

### Methods

The assay utilised a genetically modified THP-1 cell line containing a stably-transfected beta-lactamase reporter gene under control of the NFkB response element and GeneBLAzer® beta-lactamase technology (Invitrogen Corp). The cells respond to stimulation with TNFα, causing activation of the NFκB signaling pathway. Co-incubation of cells with TNFα and test material allows quantitative determination of the ability of test material to inhibit TNFa-stimulated beta-lactamase production. An inflammatory index was calculated as the ratio of beta-lactamase product to beta-lactamase substrate.

THP-1 cells were seeded into wells of a 96-well plate in the presence of RPMI 1640 medium. TNFα was added to each well to give a final concentration of 7.5 ng/ml and dialyzed bovine serum was added. Compounds dissolved in DMSO were then added. Each plate also contained no-cell controls, no-serum controls and serum controls. Plates were incubated for 5 h at 37° C. to allow for NFκB-stimulated beta-lactamase production. LiveBLAzer™ FRET B/G Substrate (CCF4-AM) substrate was then added to assay. Once CCFA-AM enters a cell, it is converted to negatively charged CCF4 by endogenous esterases. Excitation of this substrate at 409 nm leads to efficient FRET between the coumarin and fluorescein moieties, resulting in a green fluorescence detectable at 530 nm. The presence of beta-lactamase leads to cleavage of CCF4 and results in a loss of FRET, resulting in a robust blue fluorescent signal detectable at 460 nm. Thus, activity of beta-lactamase (a marker of NFκB-promoter activity) is measured as a product to substrate ratio (blue/green fluorescence ratio: 460 nm/530 nm).

### Results

It was found that 30 μM was the optimal concentration at which to compare the NFkB-inhibitory activity of compounds because at concentrations greater than 50 μM, cell viability was reduced. MTT is bioreduced by viable cells into a coloured formazan product that is soluble in DMSO. Thus the quantity of formazan product is directly proportional to the number of living cells in culture, and can be measured using a spectrophotometer at 570 nm. These compounds reduce MTT in the absence of cells, resulting in a change in substrate colour from yellow to purple. This activity is dependent on the reducing power of each compound and was measured at 5 concentrations (data not shown). The ability of compounds to reduce MTT was not related to their ability to inhibit NFkB-activity.

Data are presented following incubation with test compound at 30 μM as shown in FIG. 1. Cpd. 14, Cpd. 18, Cpd. 19, Cpd. 20 significantly inhibited NFκB promoter activity, independently of cell cytotoxicity. At this concentration, some of the test compounds reduced viability of the THP-1 cells. In particular, Cpd. 16 and Cpd. 21 reduced it by ˜10%.

### 2.1.2 Effect on Eicosanoid Synthesis

Eicosanoids, products of the metabolism various fatty acids, the main one of which is arachidonic acid (AA) are involved in both normal physiology and inflammatory responses (vasodilation, coagulation, pain and fever). There are four main families of eicosanoids—the prostaglandins, prostacyclins and the thromboxanes (known collectively as the prostanoids) and the leukotrienes. Two families of enzymes catalyse eicosanoid production:
COX, which generates the prostanoids. COX-1 is responsible for basal prostanoid synthesis, while COX-2 is important in the inflammatory response.
LO which generates the leukotrienes.

Prostanoid synthesis, and thus inflammation can be reduced by inhibiting COX, as is seen with the most prevalent class of anti-inflammatory agents, the NSAIDs (non-steroidal anti-inflammatory drugs). The following assays examine the effects of test compounds for their ability to reduce the synthesis of PGE₂ and TXB₂ produced in response to the inflammatory stimulus of lipopolysaccharide (LPS) in various cell systems.

NSAIDs have been an important therapy in the treatment of a large number of cutaneous pathologies, including psoriasis for many years. Recent studies link prostaglandin to cutaneous carcinogenesis, thus potentially expanding the use of NSAIDs to the treatment and prevention of non-melanoma skin cancer. Leukotrienes play a key role in inflammatory reactions of the skin, and in vivo and in vitro data suggest that their inhibition may have efficacy in atopic dermatitis, psoriasis and bullous dermatoses.

### 2.1.2.1 Prostanoid Synthesis in Human Monocytes

### Methods

Human peripheral blood monocytes were isolated from buffy coats by lymphoprep gradient separation followed by counter-current centrifugal elutriation. Test compounds were dissolved in DMSO and added to fresh monocytes to achieve concentrations of 0, 10 and 100 μM. After 30 min, lipopolysaccharide (LPS) was added to achieve a final concentration of 200 ng/mL. After incubation for 18 hrs at 37° C. in 5% CO₂, supernatants were removed and PGE₂ and TXB₂ (the stable hydrolysis product of TXA₂) production were measured by radioimmunoassay (RIA). ANOVA followed by Newman-Keuls multiple comparisons test was used to examine differences between doses and the control values.

### Results

Data from test compounds used at 10 μM are presented in FIGS. 2 and 3. A statistical significance level of 0.05 was used and differences from control values are indicated by an asterisk (*). The effect of test compounds on cell viability was not examined.

Similar patterns of inhibition for production of both PGE₂ and TXA₂ suggest that a compound is COX inhibitor. On that basis, all compounds demonstrated some degree of COX inhibition. Of those compounds, Cpd. 14, Cpd. 16, Cpd. 17, Cpd. 18, Cpd. 19 and Cpd. 20 demonstrated significant COX inhibitory activity in this assay.

### 2.1.2.2 Prostanoid Synthesis in a Murine Macrophage Cell Line

### Methods

The mouse macrophage cell line RAW 264.7 was cultured in DMEM supplemented with foetal bovine serum (FBS), 2 mM glutamine and 50 U/ml penicillin/streptomycin (pen/strep). Cells were treated with either test compound (in 0.025% DMSO) or vehicle alone, and added one hour before 50 ng/ml LPS. After incubation for 24 hrs, culture media was collected for PGE₂ or TXB₂ measurement by ELISA (Cayman Chemical). Data were analysed using a one way ANOVA with a Dunnett's post test to compare the various concentrations of test compounds with vehicle control (GraphPad Prism).

### Results

Because some of the compounds affected cell viability at higher concentrations, data are presented using 1 μM of test compound. All compounds substantially reduced the synthesis of PGE₂ and TXB₂ as shown in FIGS. 4 and 5. This occurred in the absence of a reduction in cell numbers due to cytotoxicity.

### 2.1.2.3 Effect on Lipoxygenase

### Background

Leukotrienes (LTs) are eicosanoids. Unlike the PGs and the TXs, which are products of the COX pathway, LTs are products of the 5-lipoxygenase (5-LO) pathway. LTs play a role in allergic and inflammatory diseases, causing increased vascular permeability, vasodilation, smooth muscle contraction, and are potent chemotactic agents. Moreover, inhibition of 5-LO indirectly reduces the expression of TNFα.

There is now much evidence for the involvement of LTs in atherosclerosis. All components involved in LT biosynthesis including 5-LO and LTB₄ are highly expressed in atherosclerotic plaques, which also have the capacity to produce LTB₄ ex vivo. LTB₄ also contributes to the involvement of LDL in atherosclerotic lesions—LTB₄ is a chemoattractant for monocytes and might initiate their recruitment, and oxidized lipids that are agonists for LTB₄ receptors might also initiate monocyte recruitment. Incubation with LDL increases LTB₄ release by neutrophils, and oxidised LDL enhanced LTB₄ production to an even greater extent than native LDL.

Currently, the mechanism LO in atherogenesis is not fully understood. There is controversy over the involvement of 5-LO and LTB₄ versus an alternative LO, 12/15-LO in the development of atherosclerosis. (12/15-LO catalyzes the transformation of free arachidonic acid to 12-HPETE and 15-HPETE. These products are reduced to the corresponding hydroxy derivatives 12-HETE and 15-HETE by cellular peroxidases. Mice make predominantly 12-HETE whereas humans produce mainly 15-HETE.) 12/15-LO and 5-LO cascades play central roles in LDL oxidation and LT biosynthesis respectively. However, 5-LO-derived LTB₄ appears to influence early atherosclerotic events in vitro and in mouse studies perhaps by mediating monocyte adhesion and recruitment via monocyte chemoattractant protein-1 (MCP-1).

LTs have also been implicated in the pathogenesis of osteoarthritis. The subchondral osteoblasts in an osteoarthritic joint can synthesise LTB₄, indicating a role of LTs in the bone remodeling associated with osteoarthritis.

Whilst the effect of leukotriene inhibitors in psoriasis in early clinical trials were disappointing, their potential has been re-examined more recently. In vitro and in vivo data have demonstrated that leukotrienes play a key role in skin inflammation, suggesting that LO inhibition may be a useful therapeutic strategy in psoriasis, particularly in combination with other agents.

LTB₄ and LTC₄ levels in tears are significantly higher in patients with allergic conjunctivitis than controls, suggesting a role for LTs in ocular allergic disorders. This is confirmed by the hypothesis that efficacy of the antihistamine mizolastine is due in part to its dual 5-LO-inhibitory activity.

### Methods

The pathway for LTB₄ synthesis involves initial release of AA from phospholipids by a Ca-dependent PLA₂. The free AA is then oxygenated at by 5-LO (requiring enzyme activation by FLAP) to generate an epoxide intermediate (LTA₄). LTA₄ is then converted to LTB₄ by LTA4 hydrolase. LTB₄ is metabolised (and deactivated) by a cytochrome P-(CYP) 450 ω-hydrolase to produce 20-hydroxy and 20-carboxy metabolites. These metabolites are also measured in the HPLC assay.

Neutrophils were isolated from citrated human venous blood to >90% purity by centrifugation through Ficoll, dextran sedimentation and lysis of erythrocytes. Cells were washed in HEPES buffered Hanks solution (HBHS) and then suspended at 4.5 million cells/mL in HBHS containing 0.1% bovine serum albumin (HBHS+BSA).

Experiments had been carried out previously to optimise the stimulation of neutrophils by calcium ionophore. At 37° C., cells were incubated with the test compound in 10 μL DMSO for 5 min before addition of 100 μL of 25 ng/μL calcium ionophore (free acid form, Sigma) with 0.5% DMSO in HBHS+BSA. The cells were incubated for 10 min then pelleted by centrifugation at 4° C. for 5 min and the cell free supernatant used to quantitate the levels of LTB₄ and its metabolites.

To each 900 μL aliquot of the supernatant, 25 μL of prostaglandin B₂ (PGB₂) 2.5 ng/μL in ethanol was added as internal standard. The solution was acidified to pH≦3 with 2M formic acid and the mixture extracted with 2 mL ethyl acetate and vigorous vortexing. The organic layer was collected and dried under nitrogen in a glass vial before reconstituted in 50 μL of the reconstitution solution (water:methanol:acetonitrile at 2:1:1).

Analysis was carried out using an HPLC system with a 125-4 LiChrospher®100 RP-18 (5 μm) column (Agilent Technologies) and a gradient system adapted from a published method to separate LTB₄, its oxidation products 20-hydroxy LTB₄ (20-OH-LTB₄) and 20-carboxy LTB₄ (20-COOH-LTB₄), as well as PGB₂. At 1 ml/min flow rate, a combination of three different mobile phase solutions was used:

A (water:methanol:acetonitrile:trifluoroacetic acid at 80:40:40:0.1, pH=3 with Et₃N),
B (methanol:acetonitrile at 1:1), and
C (methanol).

UV absorbance was monitored at 270 nm, and LTB₄ and its metabolites were quantitated by comparison of peak areas with that of internal standard and a standard curve prepared earlier.

### Results

Cpd. 13, Cpd. 17 and Cpd. 21 were not examined in these assays. All compounds examined were very active in inhibiting the synthesis of LTB₄ and its metabolites.

TABLE 1
Effect of test compounds on synthesis of LTB₄ by neutrophils
(IC₅₀ - μM)

| Compound | (IC₅₀ - μM) |
|---|---|
| Cpd. 14 | <0.1 |
| Cpd. 16 | 2.2 |
| Cpd. 18 | 0.5 |
| Cpd. 19 | 0.3 |
| Cpd. 20 | 0.9 |
| Cpd. 21 | ND |

The maximum release of LTB4, 20-OH-LTB4 and 20-COOH-LTB4 produced by the active test compound was compared to that of vehicle control as shown in FIG. 6.

Overall the cell viability was around 75%-85%, using an aliquot of the reaction mixture and incubation cells with the test compound for 5 minutes. Cell viability was of neutrophils incubated with test compounds was similar to that of controls.

### 2.1.3 Effect on Synthesis of TNFα

Tumor necrosis factor-alpha (TNFα) is a cytokine involved in systemic inflammation and is one of the cytokines that mediate the acute phase reaction. TNFα can be produced by macrophages, T cells, mast cells, neutrophils, dendritic cells, keratinocytes and endothelial cells when exposed to inflammatory stimuli. TNFα also increases production of other pro-inflammatory molecules (e.g. IL-1, IL-6, IL-8, NFκB) and adhesion molecule expression. Consequently, it is central to many inflammatory diseases, and its inhibition is therefore an anti-inflammatory strategy.

TNFα is produced in the heart by both cardiac myocytes and resident macrophages and is considered to be involved in the triggering and perpetuation of atherosclerosis.

TNFα is also involved in the metabolic syndrome. Obesity induces an inflammatory state, due in part to adipose cell enlargement and dysregulation. This is mediated in particular by the effect of TNFα on pre-adipocytes. TNFα induces oxidative stress, causing an increase in oxidized low-density lipoprotein and dyslipidaemia, glucose intolerance, insulin resistance, hypertension, endothelial dysfunction, and atherogenesis. In fact, patients with RA have accelerated atherosclerosis, considered to be due to their cumulative inflammatory burden overall.

TNFα is the major pro-inflammatory cytokine expressed in the inflamed joints of patients with RA. Anti-TNFα therapy (as monoclonal antibodies administered via intravenous infusion) is a fully-validated treatment modality for RA, ankylosing spondylitis, psoriatic arthritis, psoriasis and inflammatory bowel disease.

TNFα also plays a major role in the pathogenesis of psoriasis. TNFα expression is increased in psoriatic lesions, higher levels of TNFα have been found in psoriasis-affected skin compared with normal skin, and the amount of TNFα in the serum of psoriasis patients correlates with disease severity. TNFα also promotes angiogenesis contributing to the increased vascularity of psoriatic lesions. TNFα is also involved in the pathogenesis of atopic dermatitis and pemphigus vulgaris, and there are many reports describing the successful use of the anti-TNFα biologics to treat otherwise non-responsive disease.

Anti-TNFα therapy (as monoclonal antibodies administered via intravenous infusion) is a fully-validated treatment modality for many disease including rheumatoid arthritis and inflammatory bowel disease. Preliminary data suggest that it may also be a suitable modality for non-infectious uveitis and perhaps diabetic macular oedema. Anti-TNFα antibodies have also been effective in a murine model of AMD.

### 2.1.3.1 Synthesis of TNFα in Human Monocytes

### Methods

Test compounds (in DMSO) at 100, 10, and 1 μmol/ were incubated with monocytes at 37° C. for 30 min after which LPS was added (200 ng/ml) and cells were incubated in triplicate for 18 h at 37° C. in 5% CO₂. After 1 h, supernatants were removed and TNFα measured by ELISA previously described. The results are shown in FIG. 7.

### Results

At 100 μM, Cpd. 14, Cpd. 18 and Cpd. 19 inhibited the synthesis of TNFα. Cell viability was not measured in this assay. A statistical significance level of 0.05 was used and differences from control values are indicated by an asterisk (*).

### 2.1.3.2 Synthesis of TNFα in a Murine Macrophage Cell Line

### Methods

Subconfluent RAW 264.7 cells were seeded into 24-well plates at 5×10⁵ cells per well and allowed to adhere for 1 hr. Cells were then treated either test compound (in 0.025% DMSO) or vehicle alone, and incubated for 1 hr. LPS 50 ng/ml (LPS—Sigma-Aldrich) was then added. After incubation for 16 hrs, culture media was collected and stored at −80° C. TNFα measurement using an ELISA (Becton Dickinson).

### Results

TNFα was also induced in this system by some of the compounds. Data from test compounds used at 10 μM are presented in FIG. 8. Cpd. 18, Cpd. 19 and Cpd. 21 inhibited TNFα. This effect occurred in the absence of a reduction in cell numbers due to cytotoxicity.

### 2.1.4 Effect on Nitric Oxide Production in a Murine Macrophage Cell Line

Nitric oxide (NO), a molecular messenger synthesized by nitric oxide synthase (NOS) from L-arginine and molecular oxygen, is involved in a number of physiological and pathological processes. Three structurally distinct isoforms of NOS have been identified: endothelial (eNOS), inducible (INOS) and neuronal (nNOS). The site of NO release impacts significantly on its net function and structural impact. Overproduction of NO by mononuclear cells and macrophages in response to iNOS, has been implicated in various inflammatory processes, whereas NO produced by endothelial cells in response to eNOS has a physiological role in maintaining vascular tone.

NO production is involved in the pathogenesis of all of the target diseases. It becomes disrupted during atherosclerosis where NO modulates COX activity via formation of the powerful oxidant peroxynitrite (ONOO⁻), resulting in changed eicosanoid production. iNOS is also important in insulin resistance—obesity is associated with increased iNOS expression in insulin-sensitive tissues in rodents and humans and inhibition of INOS ameliorates obesity-induced insulin resistance. NO production is also increased in arthritic joints and inhibitors of NO synthesis ameliorate experimentally-induced arthritis. NO contributes to T cell dysfunction in RA by altering multiple signaling pathways in T cells.

NO is produced by iNOS in keratinocytes, fibroblasts, Langerhans cells and other dendritic cells, and is reported to be involved in skin inflammatory and immune responses such as contact dermatitis and atopic dermatitis. The relationship of iNOS to psoriasis is less well understood. Whilst there is increased expression and production of iNOS in psoriatic skin, NO inhibits cellular proliferation, and abnormally low NO synthesis is thought to contribute to the pathogenesis of psoriasis. Pemphigus patients display increased serum NO levels that are associated with increased iNOS expression in the affected skin.

NO is an important mediator of homeostatic processes in the eye, such as regulation of aqueous humor dynamics, retinal neurotransmission and phototransduction. NO generation is associated with inflammatory diseases (uveitis, retinitis), and degenerative diseases (glaucoma and AMD) and increased levels in the aqueous and vitreous humors are found in diabetes, which is thought to be a contributing factor in glucose-induced cataract formation. In ‘dry eye’, the expression of INOS in conjunctival epithelium correlates with disease severity. NO is an important factor in the induction and progress of the allergic reaction to the ocular surface, and its inhibition is considered a therapeutic strategy in allergic conjunctivitis.

### Methods

Nitrite concentration is a quantitative indicator of NO production and was determined by the Griess Reaction. Briefly, 100 μL of Griess reagent was added to 50 μL of each supernatant in duplicate in two separate assays, run as for the examination of PGE₂ etc. The absorbance at 550 nm was measured, and nitrite concentrations were determined against a standard curve of sodium nitrite. Data were analysed using an unpaired two-tailed t test (GraphPad Prism).

### Results

Treatment with all compounds tested at 10 μM inhibited the production of nitrite by macrophages stimulated by LPS, most of them statistically significantly as shown in FIG. 9. This finding confirms their anti-inflammatory activity. This effect occurred in the absence of a reduction in cell numbers due to cytotoxicity.

### 2.1.5 Effect on Adhesion Molecule Expression

Central to the inflammatory response is the migration of leucocytes from the microvasculature to the site of inflammation. For example, early atherosclerosis involves the recruitment of inflammatory cells from the circulation and their transendothelial migration. This process is predominantly mediated by cellular adhesion molecules, which are expressed on the vascular endothelium and on circulating leukocytes in response to several inflammatory stimuli. Selectins (P, E and L) are involved in the rolling and tethering of leukocytes on the vascular wall. Intercellular adhesion molecules (ICAMs) and vascular cell adhesion molecules (VCAM-1), as well as some of the integrins, induce firm adhesion of inflammatory cells at the vascular surface. VCAM-1 expression is restricted to lesions and lesion-predisposed regions whilst ICAM-1 expression is broader and extends into uninvolved regions.

The pathogenesis of common dermatoses such as psoriasis and atopic dermatitis includes the tissue-selective recruitment of lymphocytes to the skin by adhesion to the endothelial lining, extravasation, migration through the connective tissue, and, finally, localisation of a subpopulation of lymphocytes into the epidermis.

For diseases with a prominent inflammatory response such as psoriasis or RA, interference with leukocyte adhesion and/or emigration is a recognised therapeutic strategy.

There is increased adhesion molecule expression the conjunctival epithelium in ‘dry eye’ and higher levels of circulating ICAM-1 with AMD. Higher levels of ICAM and VCAM were found in the aqueous humor from patients with uveitis than in that from controls. In allergic conjunctivitis, VCAM-1 mediates the infiltration and activation of eosinophils and Th2 cells. ICAM-1 and VCAM-1 are upregulated in the conjunctiva of diabetic patients with and without retinopathy.

### Methods

Inhibition of TNFα-stimulated endothelial cell activation by compounds was assessed by measuring surface expression of cell adhesion molecules with an ELISA. Human arterial endothelial cells (HAECs) in growth medium (Cell Applications Inc.) were seeded into 96-well plates at a density of 10,000 cells per well. Plates were incubated overnight at 37° C. in a humidified incubator to allow for cells to become confluent. On the morning of the experiment, TNFα (10 μl, 2 ng/ml) was added to each well, which contained 100 μl of medium. Compounds were diluted in DMSO-containing medium (2.5% DMSO) to give a compound concentration of 100 and 300 μM. Compounds were added to wells so that final concentrations were 10 and 30 μM. DMSO-containing medium alone was added to zero concentration control wells. All samples were measured in quadruplicate (4 wells per treatment).

After incubation with compound for 4 hours, medium was removed and cells were probed with either non-specific IgG or specific mouse antibodies against VCAM, ICAM or E-selectin (BD Biosciences—0.1 μg in 100 μL buffered saline with 10% heat-inactivated human serum). Adhesion molecule expression was detected by addition of sheep anti-mouse antibody/horseradish peroxidase conjugate. Plates were allowed to stand for 30 minutes—monolayers were then washed, and sheep anti-mouse antibody/horseradish peroxidase conjugate (1:500 in 100 μL HBSS with 10% heat-inactivated human serum and 0.05% Tween 20) was added and left for 30 minutes. After further washing, 150 μL ABTS substrate (Kirkegaard and Perry Laboratories) was added to each well and allowed to develop for 15 minutes. Optical density was measured at 405 nm with an ELISA reader (Titertek Multiscan, Flow Laboratories).

### Results

The results are shown in FIG. 10. For some compounds, HAEC viability affected at concentration greater than 10 μM, so that concentration was selected as the most appropriate concentration for comparing the activity of compounds in this assay.

Cpd. 18, Cpd. 19 and Cpd. 20 significantly inhibited TNFα-induced VCAM expression. Cpd. 16 had intermediate activity, but that may have been due to reduced cell viability.

None of the compounds affected TNFα-induced ICAM expression.

Cpd. 18 and Cpd. 20 inhibited TNFα-induced E-selectin expression.

### 2.2 Anti-Oxidant Activity

Reactive oxygen species (ROS) including oxygen ions, peroxides and superoxides are free radicals, small molecules which are capable of damaging cells and DNA via oxidative stress. ROS can initiate lipid peroxidation, direct inhibition of mitochondrial respiratory chain enzymes, inactivation of glyceraldehyde 3-phosphate dehydrogenase, inhibition of membrane sodium/potassium ATP-ase activity, inactivation of membrane sodium channels, and other oxidative modifications of proteins, all of which play a role in the pathophysiology of inflammation. Antioxidants prevent the formation of free radicals, so compounds with antioxidant capabilities can potentially reduce inflammation.

Atherosclerosis is a specific chronic inflammatory response. Superoxide anions (O₂⁻), a form of ROS, and promote neointimal growth by specifically augmenting neointimal smooth muscle cell proliferation following arterial injury. Isoflavones possess antioxidant activity, and treatment with an isoflavone metabolite attenuated increases in both ROS and neointimal proliferation associated with balloon angioplasty in rabbits. Oxidation of lipoproteins, produced by ROS, are thought to provoke a number of changes in cell functions that promote atherogenesis. Oxidized low density lipoprotein (OxLDL) is also pro-inflammatory, it can cause endothelial dysfunction and it readily accumulates within the arterial wall. The anti-oxidant Probucol has demonstrated strong activity in reducing the progression of carotid atherosclerosis in clinical trials.

It has been hypothesised that the many factors causing insulin resistance are mediated via the generation of abnormal amounts of ROS, and one of the defects in metabolic syndrome and its associated diseases is excess cellular oxidative stress.

RA is associated with a disturbed intracellular ‘redox equilibrium’, the balance between oxidizing and reducing species. ROS are intracellular signalling molecules that amplify the synovial inflammatory-proliferative response. Repetitive cycles of hypoxia and reoxygenation associated with changes in synovial perfusion are postulated to activate NFκB, which then stimulates the expression of genes which maintain synovitis.

The skin is exposed to endogenous and environmental pro-oxidant agents, leading to the generation ROS. The resulting oxidative stress damages proteins, lipids, and DNA. ROS are known to play a role in the pathogenesis of psoriasis and atopic dermatitis, where there is an imbalance in the redox balance. Monocytes and mast cells from patients with atopic dermatitis generate ROS which may act as secondary messengers in the induction of other biological responses. In pemphigus vulgaris, activated neutrophils increase the production of ROS.

Oxidative stress to the eye has been hypothesised to play a role glaucoma, cataract, uveitis and AMD. The increased capillary permeability and angiogenesis causing the vision loss of diabetic retinopathy is due in part to oxidative stress.

Some of the test compounds have been demonstrated in a number of assays to have robust antioxidant activity.

### 2.2.1 Effect on Free Radical Scavenging

The antioxidant (free radical trapping) activity of test compounds was assessed using the stable free radical compound 2,2-diphenyl-1-picrylhydrazyl (DPPH). A stock solution of DPPH was prepared at a concentration of 0.1 mM in ethanol and mixed for 10 minutes prior to use. Test compounds at a concentration of 100 μM were reacted with DPPH for 20 minutes, after which time the absorbance at 517 nm was determined and the change in absorbance compared to a reagent blank (DPPH with ethanol alone). A dose response curve was produced for those compounds with free radical scavenging activity (ΔAbs>0.3) at 100 μM. The IC₅₀ value was estimated as the concentration of test compound that caused a 0.6 change in absorbance (with 1.2 absorbance units representing total scavenging of the DPPH radical).

TABLE 2
Free radical scavenging ability of test compounds - EC₅₀ (μM)

| Compound | EC₅₀ (μM) |
|---|---|
| Cpd. 13 | 94 |
| Cpd. 14 | 47 |
| Cpd. 16 | 45 |
| Cpd. 18 | 48 |
| Cpd. 19 | 74 |
| Cpd. 20 | 24 |
| Cpd. 21 | 43 |

In this assay, all compounds demonstrated the ability to scavenge free radicals. Cpd. 13 had limited activity.

### 2.2.2 Effect on Peroxyl Radical-Induced Red Blood Cell (RBC) Lysis

This assay utilises an intact cell system which may reflect more accurately the ability of test compounds to act as an antioxidant in the presence of metabolic processes (e.g. NADPH regeneration, re-cycling of antioxidants) which cannot be ascertained in the previous systems.

### Methods

Freshly collected heparinised venous blood (10 ml, on ice) was aliquotted into 1.8 ml sterile eppendorf tubes and centrifuged for 10 minutes at 2600 rpm at 4° C. Plasma and buffy coat layers were removed (approximately 900 μl) and packed red blood cells (RBC) were then washed by the addition of 900 μl of sterile, ice cold PBS. This washing procedure was repeated twice. Packed RBC were resuspended by the addition of 900 μl of ice-cold, sterile PBS (and termed RBC stock). RBC stocks were stored at 4° C. for a maximum of three days. All working suspensions of RBC were prepared fresh daily by diluting 200 μl of RBC stock into 10 ml of ice-cold, sterile PBS and 50 μl added to each well.

The free radical generator AAPH (1.22 gm) was dissolved in 7.5 ml of PBS to yield a 4× stock at 600 mM and 50 μl aliquots (final concentration of 150 mM) were then added to each well to initiate the lysis assay. Test compounds were examined at 100, 30 and 10M (in DMSO 0.25%). Appropriate controls were included in each experiment. Peroxyl-induced RBC lysis assays were performed in 96-flat bottom well microtitre plates with a total volume of 200 μl per well. Turbidity of RBC suspensions were monitored using a Tecan microplate reader at 690 nm (37° C.) with gentle vortexing. Assays were performed in quadruplicate and readings were taken every 5 minutes over 5 hours. RBC lysis curves were constructed by plotting absorbance (mean of 4 readings) against time. Time to half-lysis was calculated by taking the highest absorbance reading (no lysis) and the lowest absorbance reading (maximum lysis). The sum of these two readings divided by two gave the absorbance at half-lysis. Simple regression analysis was used to calculate the time at which half-lysis absorbance occurred.

### Results

All compounds tested demonstrated considerable antioxidant activity by delaying the AAPH-induced time to half-lysis of red blood cells.

TABLE 3
Time taken to reach half-lysis following incubation with
test compounds at 10 μM (min)

| Compound | time (min) |
|---|---|
| vehicle | 40.0 |
| Cpd. 14 | 134.7 |
| Cpd. 16 | 164.3 |
| Cpd. 18 | 107.7 |
| Cpd. 19 | 111.6 |
| Cpd. 20 | 122.0 |
| Cpd. 21 | 140.8 |

### 2.2.3 Effect on Extracellular Superoxide Production

The effect of test compounds on the production of superoxide was examined.

### Methods

The human promyeloblast cell line HL-60 can be differentiated into neutrophil-like cells, which then produce ROS when activated. HL-60 cells were grown in RPMI-1640 medium containing glutamine and supplemented with FBS 20%. They were differentiated by culturing for 6 days in medium containing DMSO 1.25%, after which they were washed, centrifuged and incubated at 37° C. for 5 minutes with cytochalasin before transfer to PBS containing cytochrome C and test compound. After a 5 minute incubation, phorbol myristate acetate (PMA) was added to activate the HL-60 cells, which were then incubated for a further 10 minutes. The cells were then pelleted by centrifugation, and the change in absorbance due to reduction of cytochrome C in the cell-free supernatant was measured at 550 nm. The increase in absorbance is a direct measure of extracellular superoxide production by the cells, and a reduction in those samples incubated with test compounds would indicate anti-oxidant activity. Samples were examined in duplicate, and the assay done three times.

### Results

Six compounds were tested in this assay. Cpd. 14, Cpd. 19 and Cpd. 21 showed a trend towards inhibition, whereas Cpd. 16 significantly inhibited superoxide production at the relatively high concentration of 100 μM. None were active at the lower concentrations of 0.1 μM and 1.0 μM. Cpd. 20 was not active.

TABLE 4
Effect of test compounds on extracellular superoxide
production (% change compared with vehicle control)

% change

| Compound | 10 μM | 100 μM |
|---|---|---|
| Cpd. 14 | −20 | −20 |
| Cpd. 16 | −22 | −57* |
| Cpd. 18 | −24 | −36* |
| Cpd. 19 | −11 | 16 |
| Cpd. 20 | 6 | 28 |
| Cpd. 21 | −10 | −11 |

### 2.3 Effect on PPARγ Activity

PPARs (peroxisome proliferator-activated receptors) are a class of intracellular receptors that when activated, cause transcription of a number of genes that modulate carbohydrate and lipid metabolism and adipose tissue differentiation. Three types of PPARs have been identified—α, γ and δ(β). PPARγ regulates glucose and lipid homeostasis. Activation of PPARγ is anti-inflammatory and appears to exert a vasculoprotective effect by limiting endothelial dysfunction, impairing atherogenesis and preventing restenosis.

Accumulating evidence suggests that PPAR agonists possess powerful anti-atherosclerotic properties, by both directly affecting the vascular wall and indirectly affecting systemic inflammation. PPAR agonists are also used to treat metabolic syndrome, dyslipidaemia, insulin-resistance and diabetes. In humans, PPARγ agonists increase insulin sensitivity, improve the plasma lipid profile and reduce inflammation. These compounds also have direct vasoprotective effects by inhibiting inflammatory cytokines in monocytes, macrophages, endothelial cells and smooth muscle cells, the signaling of angiotensin II—a major proinflammatory and proatherogenic factor, and the migration and proliferation of VSMC. PPARγ agonists can thus cause reduction of neointimal hyperplasia in animal models.

PPARγ activation may be protective in osteoarthritis. PPARγ expression is down regulated in arthritic cartilage and PPARγ activators demonstrate anti-inflammatory and chondroprotective properties in vitro and improve the clinical course and histopathological features in experimental animal models of osteoarthritis.

The epidermis, a very active site of lipid metabolism, expresses all PPAR isoforms. Their activation stimulates keratinocyte differentiation and maintains permeability barrier homeostasis. PPAR activation is anti-inflammatory, reducing inflammation in animal models of allergic and irritant contact dermatitis. In hyperproliferative psoriatic epidermis and the skin of patients with atopic dermatitis, the expression of both PPARα and PPARγ is decreased. This suggests that PPAR activators, or compounds that positively regulate PPAR gene expression may represent novel therapeutic agents for the treatment of these dermatoses. PPARγ activators, perhaps because they also decrease TNFα production have been associated with clinical benefit in psoriasis.

PPARγ is present in in ocular endothelial cells, and in several animal models PPARγ agonists prevented choroidal and retinal neovascularisation via the inhibition of vascular endothelial growth factor (VEGF) receptor expression. PPARγ may present be a novel pharmacological target of angiostatic agents, particularly useful to treat AMD and diabetic retinopathy, as well as ocular burns.

Cpd. 13 and Cpd. 17 were not screened for PPARγ agonist activity.

### Methods

Human HEK293 cells, stably transfected with the PPARγ ligand binding domain fused with the DNA binding domain of the GAL4 protein (GAL4-PPARγ fusion protein), produce beta-lactamase when incubated with PPARγ ligands. Transfected human kidney embryonic cells (Invitrogen Inc., Carlsbad, Calif.) were seeded onto matrigel in 96-well plates and allowed to attach overnight. The following day, vehicle alone (DMSO) or test compound at 1, 5 and 10 μM was added at varying concentrations to cells and incubated for 16-18 hours. Cells were then loaded with a FRET-based fluorescent substrate to assess beta-lactamase activity. Cells were protected from light, and incubated at room temperature for 2 h. Plates were read on a fluorescence plate reader with an excitation wavelength of 409 nm and emission wavelengths of 460 nm and 530 nm. Results were expressed as a ratio of these two wavelengths after the background (cell-free control wells) had been subtracted. PPARγ activity was thus determined by measuring beta-lactamase activity as assessed by a fluorescent product to substrate ratio.

### Results

PPARγ activation, as determined by an increase in beta-lactamase activity, occurred with Cpd. 16, Cpd. 19 and Cpd. 20, suggesting that these compounds had some PPARγ agonist activity as shown in FIG. 11.

### 2.4 Immunomodulating Activity

### 2.4.1 Immunology of Targeted Diseases

The immune system is an important component of atherosclerotic inflammation. Both T- and B-lymphocytes can modulate the progression of atherogenesis, primarily through cytokine secretion and immunoglobulin production respectively.

Atherosclerotic plaques contain numerous T cells, the majority of which are CD4+ cells, although smaller numbers of CD8+ cells have been detected. Among the CD4+ cells are several subgroups, including Th1 cells which mainly secrete proinflammatory cytokines eg INFγ, and Th2 cells which may be anti-inflammatory and do not produce INFγ. The pattern of cell and cytokine involvement suggests a Th1 dominance in atherosclerotic lesions. INFγ appears to have a pro-atherogenic role—atherosclerotic lesions are increased in both INFγ^{-/-} mice and where recombinant INFγ is injected into hyperchlolesterolaemic mice. IFNγ activates macrophages (the most prominent cell type in plaques), thereby increasing their production of NO, pro-inflammatory cytokines, and pro-thrombotic and vasoactive mediators.

T cells also produce the pro-inflammatory cytokine TNFα, which can activate the NFκB pathway, in turn causing the production of ROS. TNFα also has marked metabolic effects that include the suppression of lipoprotein lipase, which leads to the accumulation of triglyceride-rich lipoproteins in the blood. Increases in both lipoproteins and the TNFα have been associated with heart disease in clinical studies.

Experimentally, B cells have been shown to be atheroprotective, because eliminating them either genetically or through splenectomy increases atherosclerosis, and this action may be because of the production of α-OxLDL antibodies. B cells can regulate the immune response directly through cytokine secretion as well. Under certain conditions, B cells are able to produce a variety of cytokines once thought to be restricted to T-cells, including IL-6, IFN-γ and TNFα. T cells are found within the actual plaque. B cells are rarely present, but they are common among the neighbouring adventitia.

IL-6 is a pro-inflammatory cytokine associated with the acute phase response. IL-6 levels are also associated with subclinical atherosclerotic lesions independently of traditional risk factors, and the influence of IL-6 on ICAM-1 secretion may play a role in this association.

Metabolic syndrome and insulin resistance have immune components, with NFκB and TNFα being the central mediators. Increasing adiposity activates inflammatory responses in fat and liver, with associated increases in the production of cytokines and chemokines. Immune cells including T cells are recruited and/or activated, causing local insulin resistance.

RA is considered to be predominately a Th1-mediated disease, although B cells and the MHC II also contribute. T cells produce pro-inflammatory cytokines including IFNγ, IL-1, IL-2, IL-6, IL-17, TNFα, as well as being involved in osteoclast activation and bone resorption. B cells also produce inflammatory cytokines, as well as autoantibodies.

The excessive growth and aberrant differentiation of keratinocytes found in psoriasis is triggered by activation of T cells, dendritic cells and various immune-related cytokines and chemokines. It is thought that skin antigens stimulate Langerhans cells which in turn activate T cells, which differentiate and undergo clonal expansion within the lymph nodes. These T cells migrate to the skin where they release a cascade of Th1 cytokines such as INFγ, IL-2 and TNFα, causing epidermal and vascular hyperproliferation.

Atopic dermatitis is a T cell-mediated disease with a Th2 cytokine pattern, at least in the initial stages. Contact dermatitis is an immune response to contact allergens, causing tissue-specific migration of effector and regulatory T cells. Pemphigus and bullous pemphigoid are both chronic autoimmune diseases, mediated by circulating autoantibodies to structural components maintaining cell-cell and cell matrix adhesion in the skin and mucous membranes.

There is increasing evidence that AMD is due to a failure in ocular immune down-regulation enabling T cell activation, and that choroidal neovascularisation may be controlled by immunosuppression. The central mechanism in the pathogenesis of allergic conjunctivitis is IgE-mediated mast cell degranulation and activation of eosinophils and T lymphocytes involving both Th1- and Th2-mediated cytokines. Likewise with ‘dry eye’, a murine model of KCS has demonstrated that the inflammation of the lachrimal duct, cornea and conjunctival epithelium is T-cell mediated, with IFNγ having a pivotal role in promoting conjunctival squamous metaplasia. One of the post-operative complications of cataract surgey in posterior capsular opacification, which is considered in part to be due to an increase in the production of IL-1 and IL-6 by lens epithelial cells.

### Methods

The effects of the test compounds on T- and B-cell proliferation and their production of IFNγ, TNFα and IL-6 were examined.

Male Skh-1 (hairless) mice, approximately six weeks old were killed by cervical dislocation. Single cell suspensions were made from the spleen and erythrocytes were lysed in buffer (0.14M NH₄Cl, 17 mM Tris, pH 7.2). The remaining splenocytes were cultured in RPMI-1640 (Gibco) supplemented with 10% (v:v) FBS, 2 mM L-glutamine, pen/strep and 50 μM 2-mercaptoethanol. Splenocytes were added to quadruplicate wells containing either the T cell mitogen concanavalin A (Con A, Sigma-Aldrich—0. 4 μg/well), the B-cell mitogen LPS (Sigma-Aldrich—1 μg/well) or no mitogen, as well as test compound at a concentration of 10 μM in DMSO. Samples were analysed after a 3 day incubation at 37° C. in 5% CO₂ in air. Cell viability was assessed by adding MTT to each well, incubating for a further 4 hrs and then developing colour with 0.04N HCl in isopropanol. Supernatant samples were frozen at −80° C. and IL-6, IFN-γ and TNFα were detected in triplicate using ELISA kits (BD Biosciences).

### Results

Cpd. 14, Cpd. 16, Cpd. 18, Cpd. 19, Cpd. 20 and Cpd. 21 were examined as shown in FIG. 12. Results for cell viability/proliferation are the average of assays from four mice; cytokine results are the average of assays from two mice each. Results are presented as % change compared to vehicle. Asterisks (*) indicate where there was a statistically significant difference from vehicle control in the assays from all mice tested.

### 2.4.2 Effect on Lymphocyte Viability and Proliferation

Cpd. 20 was suppressive to non-proliferating splenocytes, and inhibited the proliferation of both T and B cells. Cpd. 14 and to a lesser extent Cpd. 16 augmented the proliferation of resting cells, as well as T and B cells. Cpd. 18, Cpd. 19 and Cpd. 21 increased B cell proliferation by 15-20%.

### 2.4.3 Effect on INFγ Production

All compounds tested inhibited INFγ synthesis by T cells as shown in FIG. 13. Cpd. 18, Cpd. 19 and Cpd. 21 did so in the absence of cellular suppression, suggesting that they may be functionally immunosuppressive (ie immunosuppression without cytotoxicity). The marked reduction in INFγ seen with those cells treated with Cpd. 20 may be contributed to by lymphocyte suppression.

### 2.4.4 Effect on TNFα Production

All compounds tested tended to inhibit TNFα production by T cells as shown in FIG. 14. Again, Cpd. 18, Cpd. 19 and Cpd. 21 did so in the absence of toxicity. In this assay system, Cpd. 14, Cpd. 16, Cpd. 20 and Cpd. 21 induced TNFα synthesis by B cells. (Cpd. 14, Cpd. 16 and Cpd. 20 tended to do so in RAW 264.7 cells stimulated with LPS as well—see above). However Cpd. 19 inhibited TNFα in B cells without affecting cell viability and proliferation.

### 2.4.5 Effect on IL-6 Production

All compounds tested inhibited the production of IL-6 in T and to a lesser extent, B cells as shown in FIG. 15. In T cells, the effect of Cpd. 20 was most marked, but that observation would be influenced by the underlying reduction in cell numbers compared with incubation with vehicle control alone. However, Cpd. 16, Cpd. 18 and Cpd. 19 reduced IL-6 production in T cells without reducing their numbers, suggesting again that those compounds may be functionally immunosuppressive.

### 2.4.6 Summary

All compounds tested are shown to be immunosuppressive. Cpd. 20 achieved this at least in part via T and B cell cytotoxicity, whereas the other compounds are functionally immunosuppressive without being cytotoxic.

### 2.5 Vascular Activity

### 2.5.1 Effect on Proliferation of Vascular Smooth Muscle Cells

VSMC proliferation is an important step in the atherosclerotic process. The vascular remodelling in atherosclerosis involves VSMC changing from the quiescent ‘contractile’ phenotype to the active ‘synthetic’ state, where they migrate and proliferate from media to the intima, causing intimal thickening. Consequently, an agent that inhibits proliferation of VSMC is likely to have anti-atherogenic properties.

In general, the compounds were examined two or three times in each cell type. At higher doses (≧75 μM), all compounds were inhibitory to all cells/cell lines, and this effect was most probably due to cytotoxicity.

### Human Coronary Artery Smooth Muscle Cells

Human coronary smooth muscle cells (HCASMC—Clonetics) supplied at passage 3 and used for up to a further 12 population doublings, were seeded into 96 well plates at a low seeding rate, 2-5×10³ cells per well and allowed to attach and proliferate to 30-40% confluence for 24-48 hours. Prior to inoculation the medium was changed to fresh growth medium. Test compounds were prepared in growth medium and added to plates so that the final concentrations were a series from 150 μM to 0.6 μM. The cells were incubated for five days, cell number assessed using MTT and an IC₅₀ for each compound calculated.

### Human Umbilical Vein Smooth Muscle Cells

Human umbilical vein smooth muscle cells (HUVSMC), tissue explants from a male neonate (HRI— passage 2), were seeded into 96 well plates at 1×10³ cells per well, and allowed to attach for 24 hours. They were then washed twice and incubated in medium without FBS for 24-48 hours to serum-starve them. Test compounds were prepared in medium without FBS and added to the plates and incubated for one hour. Medium with FBS was added to give a final concentration of 10% and the plates incubated for 5 days until the controls were just confluent. Final analogue concentration was therefore either a series from 150 μM to 1.2 μM, or single concentrations of 10 μM. The difference in absorbance between treated cells and untreated cells was calculated using the formula, test/control*100, to obtain the percentage change caused by the test compound. As well, an IC₅₀ for each compound was calculated.

### Rat Aortic Smooth Muscle Cell Lines

The effect of test compounds a rat aortic smooth muscle cell line (A7r5) and another cell line from the media of rat aorta (A10) was examined. The methodology was the same as for HUVSMC, except that the cells were treated with compound for three days.

### Results

Relative efficacy of the test compounds can be assessed in the table below, which grades the IC₅₀ for each compound in each cell line.

TABLE 5
Summary of effect on VSMC proliferation - IC₅₀ (μM)

| | HCASMC | HUVSMC | A7r5 | A10 |
|---|---|---|---|---|
| Cpd. 14 | 54 | 64 | 33 | 10 |
| Cpd. 16 | 118 | 63 | 48 | 31 |
| Cpd. 18 | 88 | 140 | 74 | ND |
| Cpd. 19 | 63 | 34 | ND | ND |
| Cpd. 20 | 27 | 32 | 33 | ND |
| Cpd. 21 | 29 | 8 | ND | ND |

2.5.2 Effect on Expression of Endothelial Nitric Oxide Synthase (eNOS)

NOS is the enzyme which produces nitric oxide (NO) and when it does so in the vascular context (eNOS), the NO produced is vasodilatory. The induction of eNOS is therefore considered a cardioprotective strategy. NO synthesised by eNOS is the principal mediator of endothelial function—it is a potent vasodilator, it inhibits platelet aggregation, VSMC migration and proliferation, and monocyte adhesion. All these actions lead to the inhibition of both vascular negative remodelling and neointimal formation after vascular injury.

There also appears to be a link between eNOS and the metabolic syndrome: eNOS^{-/-} mice display hypertension, insulin resistance and dyslipidaemia and eNOS polymorphisms in humans are associated with hypertension, insulin resistance and diabetes.

eNOS has a role in arthritis. Based on the observation that eNOS^{-/-} mice are osteoporotic due to defective bone formation, it appears that eNOS regulates osteoblast activity and bone formation. Other studies have indicated that the NO derived from the eNOS pathway acts as a mediator of the effects of oestrogen in bone, as well as the effects of mechanical loading on the skeleton where it promotes bone formation and suppress bone resorption. Ocular blood flow is regulated by endothelial-derived NO, and it is thought that reduced expression of eNOS may contribute to diabetic retinopathy and macular edema.

Consequently, eNOS enhancement may prove to be a therapeutic strategy for atherosclerosis, metabolic syndrome, ocular inflammation and possibly arthritis.

The effect of test compounds on the expression of eNOS by HCAECs was examined.

### Methods

HAECs were grown as described above. Because cell viability was less than 100% at 30 and 100 μM, eNOS experiments were conducted at one concentration (10 μM), with exposure to test compounds for 24 hrs. After incubation, total RNA was extracted using TRI reagent (Sigma, St Louis, Mo., USA), following the manufacturer's protocol. RNA was quantitated and normalized to 100 ng/μl using the SYBR Green II assay (Molecular Probes, Eugene, Oreg., USA) before being reverse transcribed using iScript (Bio-Rad, Hercules, Calif., USA). eNOS (sense 5′-CCA TCT ACA GCT TTC CGG CGC-3′ and antisense 5′-CTC TGG GGT GGC CTT CAG CA-3′) and 18S (sense 5′-CGG CTA CCA CAT CCA AGG AA-3′ and antisense 5′-GCT GGA ATT ACC GCG GCT-3′) mRNA levels were determined by real-time PCR using iQ SYBR Green Supermix (Bio-Rad) in an iCycler iQ RealTime thermocyler detection system (Bio-Rad Laboratories). The cycling parameters were 95° C. for 30 seconds, 62° C. for 30 seconds, and 72° C. for 30 seconds for 40 cycles, and real-time data was collected at each cycle.

### Results

Test compounds were examined at a single concentration of 10 μM as shown in FIG. 16. Cpd. 14 significantly increased the expression of eNOS without affecting the viability or proliferation of the HAECs.

Subsequently, Cpd. 14 and Cpd. 18 were examined in two more assays. Cpd. 14 and Cpd. 18 significantly increased eNOS mRNA by an average of 86% and 62% respectively over vehicle control.

### 2.5.3 Effect on Endothelial Dysfunction—Vasodilatory Activity in the Rat Aortic Ring Assay

The endothelium regulates VSMC contractility by the production of relaxing and constricting factors in response to physiologic stimuli. Endothelial dysfunction is characterised by impairment of endothelium-dependent vasodilation (EDV) and by pro-coagulant/pro-inflammatory endothelial activities, so the assessment of EDV is a common parameter for testing endothelial function.

Endothelial dysfunction is integral to early atherosclerosis, and may precede structural changes and clinical manifestations.

Metabolic syndrome and insulin resistance are also associated with endothelial dysfunction such as altered patterns of blood flow regulation, vascular reactivity, microvascular density, and vascular wall mechanics, and there is some evidence to suggest that that microcirculatory abnormalities may be not only secondary but also be causal and/or contributory.

Both RA and psoriasis patients have a higher incidence of cardiovascular disease than the baseline population, manifested by atherosclerosis and the associated endothelial dysfunction. Psoriasis is a risk factor for cardiovascular diseases, so its adequate management must include the treatment of other known risk factors. In a study examining patients with psoriatic arthritis without cardiovascular risk factors or clinically evident cardiovascular disease were shown to exhibit endothelial dysfunction.

The vasodilatory capacity was examined ex vivo using the rat aortic ring assay. The addition of noradrenaline to the test bath causes the rings to contract, and if that vasoconstriction is inhibited by a test agent ie it antagonises the effect of noradrenaline, it suggests that the agent may have vasodilatory activity.

### Methods

Male Sprague-Dawley rats (250±50 g) were euthanased with 80% CO₂ and 20% O₂. The thoracic aorta was excised and quickly mounted in organ-baths as described. Full concentration-contractile curves were obtained to noradrenaline (0.1 nM-10 mM) with and without test compounds delivered at a concentration of 1 μg/ml. Experiments were repeated in n=5 different rings from 5 different animals. Only one compound at any one concentration was tested on any one ring from any one animal. Sigmoidal dose response curves were fitted for the data and the logEC₅₀ and the Ema, calculated (Prism 4, GraphPad Software). The difference in these values between the presence and absence of test compound was calculated using a two-tailed paired t test. The effects of β-oestrodiol and vehicle alone were examined as a positive and negative control respectively.

### Results

Cpd. 13 and Cpd. 20 significantly inhibited both the contractile response (logEC₅₀) of the aortic ring to noradrenaline and reduced the strength of that contractile response (Eₘₐₓ). Cpd. 18 and Cpd. 21 inhibited just the contractile response. The results suggest that several of the compounds may be potentially vasodilatory.

TABLE 6
Effect of test compounds on the contractile response to noradrenaline

| | log EC₅₀ | log EC₅₀ | | p |
|---|---|---|---|---|
| Compound | before NA | after NA | Difference | Value |
| Cpd. 13 | −8.152 | −7.733 | 0.419 | 0.002 |
| Cpd. 14 | −8.054 | −7.948 | 0.106 | 0.324 |
| Cpd. 16 | −8.132 | −8.065 | 0.067 | 0.774 |
| Cpd. 18 | −8.1 | −7.811 | 0.289 | 0.024 |
| Cpd. 19 | −8.256 | −8.207 | 0.049 | 0.742 |
| Cpd. 20 | −8.188 | −7.803 | 0.385 | 0.018 |
| Cpd. 21 | −8.367 | −7.871 | 0.496 | 0.008 |

TABLE 7
Effect of test compounds on strength of the contractile response to
noradrenaline

| | log EC₅₀ | log EC₅₀ | | p |
|---|---|---|---|---|
| Compound | before NA | after NA | Difference | Value |
| Cpd. 13 | 1.679 | 1.366 | 0.313 | 0.033 |
| Cpd. 14 | 1.732 | 1.681 | 0.051 | 0.464 |
| Cpd. 16 | 1.266 | 1.371 | −0.105 | 0.289 |
| Cpd. 18 | 1.601 | 1.498 | 0.103 | 0.140 |
| Cpd. 19 | 1.458 | 1.47 | −0.012 | 0.862 |
| Cpd. 20 | 1.962 | 1.72 | 0.242 | 0.028 |
| Cpd. 21 | 2.155 | 2.065 | 0.09 | 0.475 |

### In Vivo Activity

### 3.1 Anti-Inflammatory Activity in Murine Car Inflammation Assay

Compounds were examined for their ability to inhibit ear swelling in mice induced by the topical application of several inflammogens—AA and 4-β-phorbol 12-myristate 13-acetate (PMA). The inflammatory response due AA, the immediate precursor of the eicosanoids, is due to formation of AA metabolites via both the COX and LO pathways. AA induces an early (10-15 min) increase in both PGE₂ and LTC₄ synthesis which precedes the increase in ear thickness.

Inflammation induced by PMA involves activation of protein kinase C (PKC), a phospholipid-dependent protein enzyme which plays a key role in a range of signal induction processes. In other words, PMA is a PKC activator. PKC mediates activation of phospholipase A2, resulting in the release of free AA and the subsequent synthesis of LTs and PGs. The inflammation is primarily mediated by PGE₂, as levels of PGE₂ but not LTB₄ and LTC₄ are elevated in the ears of PMA-treated mice.

### Methods

Female BALB/c mice (ARC, WA, Australia), weighing 15-21 g, and maintained on an isoflavone-free diet (Gordon's Specialty Stock Foods, Yanderra, NSW) for at least seven days, were randomised into test groups of five or six. To reduce animal use, mice had ear swelling induced by applying AA (Sigma, Steinheim, Germany) initially, and then by phorbol 12-Myristate 13-acetate (PMA—Sigma, Mo., USA) one week later.

Test compounds were dissolved in polyethylene glycol (PEG) 400 (Sigma, St. Louis, Mo., USA):phosphate buffered saline 1:1 and injected intraperitoneally (i/p) at a dose of 25 mg/kg either 30 min prior to AA treatment or 1 hr prior to PMA treatment. Mice were anaesthetised using isoflurane (Veterinary Companies of Australia Pty Ltd, NSW, Australia) and baseline thickness of both ears was measured using a spring micrometer (Interapid, Zurich, Switzerland). Each mouse received a total of 20 μL of either AA in ethanol (50 mg/ml) or PMA in acetone (0.2 mg/ml) applied to the inner and outer surfaces of each pinna (i.e. 5 μL per ear surface). Mice were anaesthetised again to remeasure the ears at 1 hr post-AA application or at 5 hr post-PMA application.

The difference in ear swelling pre- and post-application of inflammogen for each ear was calculated. The difference in mean swelling of each test group compared to the group given vehicle alone was calculated using a two-tailed unpaired t-test (Prism 4, Graphpad Software).

### Results

Treatment with Cpd. 14, Cpd. 16, Cpd. 18 and Cpd. 19 caused a significant reduction in the ear oedema caused by the application of AA.

TABLE 8
Change in ear thickness in response to the application of AA

| | | % Change | |
|---|---|---|---|
| | Change in ear thickness | compared with | |
| Compound | (mean ± SD, ×0.01 mm) | vehicle | Significance |
| Cpd. 14 | 10.7 ± 2.9 | −34 | p = 0.0016 |
| vehicle | 16.3 ± 4.1 | | |
| Cpd. 16 | 11.6 ± 2.9 | −29 | p = 0.0074 |
| vehicle | 16.3 ± 4.1 | | |
| Cpd. 18 | 8.4 ± 3.7 | −40 | p = 0.0135 |
| vehicle | 14.0 ± 4.7 | | |
| Cpd. 19 | 9.3 ± 3.9 | −34 | p = 0.0423 |
| vehicle | 14.0 ± 4.7 | | |
| Cpd. 20 | 14.4 ± 6.1 | −11 | NS |
| vehicle | 16.1 ± 4.0 | | |
| Cpd. 21 | 14.4 ± 5.1 | −11 | NS |
| vehicle | 16.1 ± 4.0 | | |

Cpd. 19 alone significantly inhibited the inflammation caused by the application of PMA.

TABLE 9
Change in ear thickness in response to the application of PMA

| | | % Change | |
|---|---|---|---|
| | Change in ear thickness | compared with | |
| Compound | (mean ± SD, ×0.01 mm) | vehicle | Significance |
| Cpd. 14 | 27.9 ± 3.8 | +7 | NS |
| vehicle | 26.1 ± 3.9 | | |
| Cpd. 16 | 23.3 ± 4.5 | −11 | NS |
| vehicle | 26.1 ± 3.1 | | |
| Cpd. 18 | 29.6 ± 2.7 | −1 | NS |
| vehicle | 29.8 ± 4.0 | | |
| Cpd. 19 | 22.1 ± 3.9 | −26 | p < 0.0001 |
| vehicle | 29.8 ± 4.0 | | |
| Cpd. 20 | 22.7 ± 4.7 | +4 | NS |
| vehicle | 21.9 ± 11.2 | | |
| Cpd. 21 | 27.2 ± 8.6 | +24 | NS |
| vehicle | 21.9 ± 11.2 | | |

### Discussion

In this assay, compounds with 5-LO inhibitory activity are generally more effective against AA-induced oedema and compounds with COX inhibitory activity are more effective against PMA-induced oedema. Therefore, if a test compound is more effective at inhibiting AA-induced inflammation than PMA-induced inflammation, it is likely that it has LO inhibitory activity rather than COX activity.

This hypothesis is supported by the finding that specific inhibitors of COX synthesis eg ibuprofen, aspirin, piroxicam did not influence AA-induced oedema, regardless of route of administration. However, indomethacin, a COX inhibitor, does inhibit AA-induced oedema, and it has been postulated that this seeming anomaly may be due to indomethacin perhaps inhibiting phospholipase A2, particularly at high doses. Even though superoxide radicals do not appear to be produced in significant quantities with AA-induced inflammation, free radical scavengers have demonstrated strong inhibition, which suggests an alternative mechanism by which some of the compounds are active in this assay. This action may be due to direct reduction of both enzymic and non-enzymic lipid peroxidation (and hence AA metabolism), as well as to a further reduction in COX and LO because of their requirement for (hydro)-peroxides to stimulate enzymic function.

Cpd. 19, was the only compound able to inhibit both AA- and PMA-induced oedema, suggesting that it may have dual COX and LO inhibitory activity. This hypothesis was born out by the in vitro results, in which Cpd. 19 showed both strong COX and LO inhibition.

### 3.2 Anti-Inflammatory Activity in the Rat Air Pouch Assay

An alternative assay used to measure in vivo anti-inflammatory efficacy is the air pouch, which involves the repeated subcutaneous injection of air into the dorsum of rats followed 24 h later by the intrapouch injection of an inflammatory stimulus.

### Methods

Air pouches were raised on the dorsum of female Dark Agouti rats, approximately seven weeks of age. To promote the formation of a cellular membrane lining the inside of each pouch, the pouches were maintained by re-inflating on days 2 and 5 after the initial injection of air. On re-inflation, the pouch was first deflated to ensure the needle was positioned correctly, before being re-inflated with 2 mL of sterile air. Using this protocol, the pouches remained inflated until use on day 7, when they were injected with 0.5 ml of either test compound or vehicle control. After 15 min, air pouches were injected with serum-treated zymozan (500 μg). Lavage of the air pouch was performed at 4 h and leucocytes counted, after which the rats were killed, the air pouch excised and processed in formalin for histology. The sections were blinded to the person counting. Using a graticule with 100 squares and the 40× objective, the number of polymorphs were counted in the pouch lining at 10 different and non-adjacent sites. Group sizes were 5-6 rats. Data were analysed for statistical significance within each experiment and using an unpaired t test.

### Results

In general, there was concordance between the two separate measures of the extent of the inflammatory infiltrate (the numbers of leucocytes in lavage fluid and polymorphs in tissue sections). There was also concordance between the two measures for the effects of the test compounds, which strengthens the validity of each data set.

Only Cpd. 16 and Cpd. 18 were examined in this assay—both were active. The results are summarised in the table below.

TABLE 10
Effect of test compounds in the rat air pouch assay

| | Concentration | Leucocytes in | Polymorphs in |
|---|---|---|---|
| | added to Air | lavage fluid | tissue sections |
| | Pouch | (×10⁻⁷) | (per 100 squares) |
| Cpd. 16 | 1 mM | 0.53 ± 0.35 | 14.1 ± 15.8 |
| Vehicle | | 2.7 ± 1.46 | 39.2 ± 15.4 |
| | | p = 0.010 | p = 0.026 |
| Cpd. 18 | 100 μM | 0.88 ± 0.30 | 23.2 ± 10.4 |
| Vehicle | | 1.70 ± 0.89 | 32.4 ± 12.7 |
| | | p = 0.082; p = 0.041 | p = 0.228; p = 0.11 |
| | | (1-tailed) | (1-tailed) |
| Cpd. 18 | 1 mM | 0.51 ± 0.33 | 6.3 ± 1.3 |
| Vehicle | | 1.80 ± 0.99 | 28.6 ± 19.0 |
| | | p = 0.013 | p = 0.017 |

^{a}Control was the vehicle DMSO 1%/PBS.
^{b}unpaired t-test; 2-tailed

### 3.3 Anti-Inflammatory Activity in a Murine Model of UV Irradiation-Induced Skin Oedema

Acute exposure of mammalian skin to UV irradiation causes an inflammatory reaction manifested by erythema and oedema. This reaction is mediated in part by pro-inflammatory prostaglandins (PGD₂, PGE₂, PGF_{2α} and possibly PGI₂) and leucotrienes, as well as the generation of reactive free radicals and ROS.

### Methods

Groups of 4-5 female Skh:hr-1 albino mice were irradiated with 1×3 MED (minimal erythemal dose) of solar simulated UV radiation, provided by a planar bank of 6 UVA tube (Hitachi 40W F40T 10/BL, black light) and one UVB tube (Philips TL 40W/12RS) with radiation filtered through a sheet of 0.125 mm cellulose acetate (Eastman Chemical Products, Kingport, Term, USA) to give 2.96×10-4 W/cm2 UVA and 1.59×10-5 W/cm2 UVB. The distance of the UV lamp from the irradiance table surface was approximately 20 cm and temperature was controlled with an electric fan. During irradiation, the cages were rotated below the lights to reduce the variation in radiation intensity in different positions.

Either test compound (0.2 ml of a 20 μM solution) or vehicle (propylene glycol/ethanol (EtOH)/water 1:2:1) was applied to the irradiated dorsal skin at 30 min, 2 h and 4 h post-irradiation. Dorsal skin fold measurements were made with a spring micrometer prior to and at 24 hr and 48 h post-UV exposure. The difference in skin thickness pre- and post-exposure to UVR was calculated for each mouse, and the differences examined between test compound and vehicle control were analysed using an unpaired two-tailed t test.

The data are presented in tables, as well as graphically as the mean percentage inhibition of skin fold thickness, calculated as [1−[(mean % change skin thickness of test group/mean % change skin thickness of control group)×100]].

### Results

Skin fold thickening was evident at 24 hrs post-UV irradiation and peaked at 48 hrs, the last time point measured. Even though test compounds were applied only three times post-UV irradiation, and dosing was completed 20 hrs prior to the first skin fold measurement, most of the 20 compounds examined were active in reducing UV-induced inflammation, as highlighted in the tables and graphs below.

Cpd. 20 significantly inhibited inflammation at 24 hrs and Cpd. 19 significantly inhibited inflammation at 48 hrs as shown in FIG. 17.

TABLE 11
The change in dorsal skin fold thickness at 24 hrs post-irradiation

| | | difference |
|---|---|---|
| | Change in skin thickness | between test |
| Compound | (mean ± SD, ×0.01 mm) | group and vehicle |
| Vehicle | 78 ± 23 | — |
| Cpd. 14 | 77 ± 5.6 | p = 0.93 |
| Cpd. 16 | 61 ± 13 | p = 0.1539 |
| Cpd. 19 | 63 ± 8.1 | p = 0.1937 |
| Cpd. 20 | 38 ± 4 | p = 0.0043 |

TABLE 12
The change in dorsal skin fold thickness at 48 hrs post-irradiation

| | | difference |
|---|---|---|
| | Change in skin thickness | between test |
| Compound | (mean ± SD, ×0.01 mm) | group and vehicle |
| Vehicle | 147 ± 37 | — |
| Cpd. 14 | 137 ± 16 | p = 0.5452 |
| Cpd. 16 | 111 ± 15 | p = 0.0501 |
| Cpd. 19 | 96 ± 5 | p = 0.0075 |
| Cpd. 20 | 123 ± 39 | p = 0.2623 |

These results demonstrate the anti-inflammatory activity of some of the test compounds. Even though they were administered topically and after the induction of inflammation, their effects were still evident 48 hrs later.

### 3.4 Anti-Inflammatory Activity in a Murine Model of Psoriasis

UVB-irradiated mouse skin provides a model for psoriasis-like impaired cytokine, inflammatory and epidermal proliferative changes. This model examines several key biomarkers of psoriasis: the induction of the cytokines TNFα and IL-6, and the increased number of infiltrating mast cells, as indicators of inflammation, and the over expression of the adhesion molecule P-cadherin characteristic of hyperproliferation of the skin. Cpd. 18 was examined for its effect on normalising these UVB-dysregulated factors.

### 3.4.1 General Methods

Inbred, 6-8 weeks of age female C57/BL6, C3H/HeN and Skh:hr-1 (hairless) mice were fed normal stock rodent rations and tap water ad libitum. One day prior to UVB irradiation, the dorsal hair of C57/BL6 and C3H/HeN mice was removed by clipping. A single UVB tube (Phillips TL-40W/12 RS, Eindhoven, The Netherlands) emitting a spectrum of 280-365 nm with a peak emission at 310 nm, was used as the light source for the experiments. The radiation was filtered through a sheet of 0.125 mm cellulose acetate (Grafix Plastics, OH, USA) to block wavelengths below 290 nm. Irradiance was measured with an IL 1500 radiometer (International Light Inc. Newburyport, Mass.) with a UVB detector (SEE 240/UVB) calibrated to the spectral output, and recorded as 1.71×10-4 W/cm2. Groups of three female C57/BL6 and C3H/HeN mice were exposed unrestrained in their cages to 7.24 kJ/m2 UVB radiation equal to 3× the minimal erythemal dose (MED) in these haired mice (fur clipped), an exposure of approximately 70 min. Control mice were clipped of fur in the same manner but were not exposed to UVB irradiation. Groups of 3 Skh:hr-1 mice received 3.59 kJ/m2 of UVB, which is equal to 3×MED on hairless mouse skin. Temperature under the radiation source was stabilised during the exposures with surrounding curtains and an electric fan.

Cpd. 18 was dissolved as a 40 mM stock solution in DMSO, then diluted in a vehicle of propylene glycol-ethanol-water 1:2:1 to provide solutions of 0, 5, 10, 20 and 40 μM with 0.001% DMSO. The vehicle control base lotion contained 0.001% DMSO. An aliquot of 100 μL of lotion was applied to the dorsum of three mice immediately, 1 h and 2 h after UVB irradiation for 24 h time point measurements, or once daily immediately after and for up to the next four days following irradiation, for later time points, and was spread evenly using a micropipette. Control mice were restrained in the same manner but were not exposed to UVB irradiation.

3.4.2 RT-PCR Detection of TNF-α, P-Cadherin and IL 6 mRNAs

Two groups of three Skh:hr-1 mice were UVB-irradiated, with or without subsequent topical application of 20 μM Cpd. 18 lotion at 0, 1 and 2 h post-irradiation. At various time points up to 24 h post-irradiation, mice were euthanased and the mid-dorsal skin excised, snap frozen in liquid nitrogen, and stored at −80° C. until RNA extraction. The frozen skin was cut into 16 μm slices using a cryostat at −20° C. Samples of intestine and placenta were collected from normal mice for controls and RNA was extracted without cutting them into micro-slices. Total RNA was extracted, first strand DNA produced by reverse transcription, and polymerase chain reaction (RT-PCR) performed (Promega, Reverse Transcription System, Madison, Wis.). The total volume of 20 μL containing Taq polymerase (Sigma) and specific primers (Invitrogen Life Technologies, Melbourne, Australia) derived from the mouse TNF-α sequence (5′ACCCTATGCTGCTCCTGCTA3′ and 5′GGAGGGGATCAGTGTCAGAA3′, Genbank accession no BC003906), 1.5 mM MgCl2, 100 μM dNTP and reaction buffer were used to amplify the mTNF-α gene. The primers 5′ACCACTTCACAAGTCGGAGG3′ and 5′ATTCCAAGAAACCATCTGGC3′ were used to amplify the mIL-6 gene. Beta-actin sequence (5′ TGTTACCAACTGGGACGACA 3′ and 5′ GTGGACAGTGAGGCCAAGAT 3′; Genbank M12481) was used as an internal standard and PCR was performed for 35 cycles with a thermal cycler (Eppendorf AG, Hamburg, Germany) under the following conditions: initial denaturation at 95° C. for 3 min, then 94° C. for 30 s, annealing at 64-56° C. for 30 s, and extension at 72° C. for 30 s. After 34 cycles, the temperature was held at 72° C. for 10 min to allow final extension. Control RT-PCR without reverse transcriptase during RT was performed to confirm the absence of DNA contamination in the RNA samples. Final PCR products (20 μL) were electrophoresed on 2% agarose gels at 80 V for 1 h at room temperature and stained with 1 mg/mL ethidium bromide in tris-borate EDTA buffer pH 8. The bands were visualised under UV transillumination and the intensity of the expressed bands was semi-quantified using the image-analyzing software and then normalised to β-actin in the same sample.

RT-PCR identification of the cutaneous mRNA for TNFα in C3H/HeN mice revealed some expression in normal skin (‘N’), confirmed by the positive intestinal band (‘I’), that was slightly increased at 3-6 h post-UVB, after which the detectable mRNA was reduced. In Skh:hr-1 mice, IL-6 mRNA, confirmed by the positive intestinal band (‘I’), was also detected in normal skin, and was maximally increased at 24 h post-UVB. However mRNA for P-cadherin, although not evident in normal skin, was faintly detectable at 3 h, and clearly expressed at 6 h post-UVB, confirmed by the mRNA extracted from the placenta (‘P’). Immediate repeated post-irradiation applications of the Cpd. 18 markedly reduced the production of each of these mRNAs at these time points. Results are shown in FIG. 18. Image analysis of the bands in comparison with the β-actin content confirmed these trends (data not shown).

### 3.4.3 Quantification of Cutaneous TNFα by ELISA

To determine the effect of UVB irradiation on cutaneous TNFα expression, groups of three C57/BL6 and C3H/HeN mice were euthanased before and at several time points up to 72 h following UVB irradiation, with and without topical treatment with 20 μM Cpd. 18, and the mid-dorsal skin was excised. Triplicate 150 mg samples of each skin sample were transferred immediately to 3 wells of a covered 24-well tissue culture plate (Nippon Becton Dickinson Co. Ltd., Tokyo, Japan) and incubated in 1 ml of media at 37° C. in a humidified atmosphere of 5% CO₂ overnight, after which the supernatant was removed from each well and stored at −80° C. for subsequent cytokine measurement by ELISA according to the supplier (R and D Systems, Inc. Minneapolis, Nebr., USA). In brief, the wells of a 96-well ELISA plate (Corning Incorporated, Corning, N.Y.) previously coated with 100 μL of 1 μg/mL capture antibody (purified anti-mouse TNFα specific IgG, R & D Systems) were incubated with 100 μL of skin supernatant, or serially diluted standards of recombinant mouse TNFα (R & D systems) at room temperature for 2 h, followed by addition of 100 μL of 300 ng/mL detection antibody (biotinylated anti-mouse TNFα antibody; R & D systems). After washing, 100 μL of 1:200 diluted streptavidin-HRP (R & D systems) was added to each well, the plate was incubated at room temperature for 30 min, washed, and 100 μL substrate solution added (R & D systems) to each well. Colour development was stopped after 30 min with 50 μL of 1M H₂SO₄, and quantitated spectrophotometrically at 405 nm. The average concentration of TNFα in each sample was computed by four-parameter analysis using Microplate Manager Software (Bio-Rad Laboratories).

TNFα expression was not reproducibly detectable in the hairless mouse skin, but was assessed in both the C₃H/HeN and C₅₇/BL6 strains, and it was confirmed that C₃H/HeN mouse skin responded more strongly to UVB irradiation than C₅₇/BL6. Application of the Cpd. 18 lotion alone had no effect on TNFα expression (not shown).

In both strains, there was an immediate upregulation of TNFα expression following UVB irradiation, which remained elevated for at least 6 h, and decreased to the normal level by 24 h (data not shown). The 3 h post-UVB time point was selected for testing the effect of Cpd. 18, and it was clearly shown in FIG. 19 that the UVB-elevated levels in each mouse strain were significantly reduced by Cpd. 18 in a dose-dependent manner between 5-40 μM. Consistently higher levels of TNFα were measured in the C₃H/HeN mice, so that 40 μM Cpd. 18 suppressed the UVB-induced TNF-α level by 40% and 45% in C₃H/HeN and C₅₇/BL6 respectively.

### 3.4.4 Immunohistochemical Detection of IL-6 and P-Cadherin

Groups of three Skh:hr-1 mice were exposed to 3×MEdD of UVB radiation, with and without topical treatment with 20 μM Cpd. 18, and were euthanased before and at 24, 48, 72 and 96 h after irradiation, and mid-dorsal skin samples were taken for histological fixation. The samples were fixed for 6 h in Histochoice (Amresco Solon, Ohio), then processed overnight in an automated formalin-ethanol-based system (Tissue Tek VIP; Bayer Diagnostics, Ferntree Gully, Australia) and embedded into paraffin blocks. Sections of 4 μm were cut onto silane-coated slides, de-waxed and rehydrated using xylene followed by graded aqueous ethanol solutions to PBS. Endogenous peroxidase activity of the sections was quenched with 3% H₂O₂ in methanol for 10 min at room temperature and washed 3 times with PBS. The sections were then blocked with 10% skim milk in PBS for 40 min at room temperature.

The IL-6 was detected by incubation with goat anti-mouse IL-6 antibody (R & D systems) followed biotinylated anti-goat IgG (Vector Laboratories, Burl ingame, CA), before treating with StreptABComplex/HRP (biotinylated horseradish peroxidase and streptavidin; Dako Corporation, CA) for 30 min. After washing, colour was developed with 3,3′-diaminobenzidine (DAB; Kirkegaard & Perry Laboratories Inc, Gaithersburg, Md., USA. Negative staining controls omitted the primary antibody.

For P-cadherin detection, the quenched sections were immersed in 0.1 M sodium citrate (pH 6.0) at 100° C. and held at 60° C. for 1 h. Following this antigen retrieval process, non-specific binding sites for the secondary antibody were blocked by incubation for 1 h at room temperature with 100 μg/mL of goat anti-mouse IgG serum (Sigma-Aldrich) in PBS containing 1% BSA. The primary monoclonal antibody (mouse anti-human P-cadherin; Abcam Ltd., Cambridge, UK) diluted to 100 μg/mL was added and sections incubated for 2 h. After washing 3 times with PBS, 100 μg/mL of peroxidase-conjugated secondary antibody, rabbit anti-mouse IgG (Vector Laboratories) in PBS containing 1% foetal bovine serum (Life Technologies, Melbourne, Australia), was added for 1 h. Subsequently the sections were washed, the colour developed by adding DAB, then were counter-stained with haematoxylin as described above. The primary antibody was replaced with PBS for the negative staining control.

The stained sections were examined by light microscopy at 20× magnification and images were captured digitally with a Sony Hyper HAD colour video camera (Sony, Tokyo, Japan). Stain intensity was analysed using a Leica Q500 MC image processing and analysis system (Leica, Cambridge, UK) and semi-quantitated in arbitrary image analysis units as the average intensity of 15 sequential fields across the skin section for each of 3 mice per treatment group. Statistical significance of the differences between the treatments was obtained using Student's t test.

Expression of IL-6 in the skin of the mice was examined immunohistochemically before and at 24, 48, 72 and 96 h after UVB irradiation. An increase in IL-6 staining was observed by 24 h and reached a maximum approximately 18-fold increase at 72 h. Therefore this time point was selected to test the effect of Cpd. 18. The IL-6 expression at 72 h was diffuse in the epidermal layer, being most intense in the upper epidermal strata, and peak expression coincided with the maximum thickness of this epidermal layer, and with discrete dermal cells also found to be immunopositive.

Topical treatment with Cpd. 18 significantly reduced the UVB-induced levels of IL-6 immunopositivity in the epidermis at 72 h, almost completely abrogating the IL-6 staining as shown in FIG. 20. Only occasional IL-6-positive cells remained in the dermis.

Semi-quantitative image analysis as shown in FIG. 21 suggested that topical treatment with the isoflavonoids alone may have slightly induced IL-6 expression. However this was probably insignificant biologically.

Immunohistochemical staining indicated that P-cadherin was detectable at a very faint level only in normal Skh:hr-1 skin as shown in FIG. 22.

Topical treatment with 20 μM Cpd. 18 alone had no effect on P-cadherin expression in the skin as shown in FIG. 23. However, irradiation with UVB induced P-cadherin expression strongly in cells of the epidermal basal layer of Skh:hr-1 mice maximally at 72 h, with positive staining observed in the nucleus. Positive cell counts showed that there was an approximately 6-fold increase following UVB exposure. Cpd. 18 markedly reduced the UVB-induction of P-cadherin, reducing the positive cells by approximately 50%.

### 3.4.5 Histochemical Identification of Mast Cells

Groups of 3 Skh:hr-1 mice were exposed to 3×MEdD of UVB radiation, with and without topical treatments of 20 μM Cpd. 18 immediately and at 24 and 48 h post-UVB. The mice were euthanased and, and mid-dorsal skin samples were taken before and at 72 h after irradiation for histological fixation. The samples were fixed for 6 h in Histochoice and processed and wax-embedded as described above. Sections of 5 μm were cut for histochemical identification of mast cells, by staining with 0.1% toluidine blue solution in McIlvane buffer, pH 3. Positively stained cells with a diameter of 10 μm or more in the dermal compartment were counted under light microscopy (Olympus UPlanApo, Japan) using a at 20× magnification, in 10 sequential non-overlapping fields for each of 3 mice per treatment group, and the average mast cell number per field was obtained. Statistical significance of the differences between groups was determined using Student's t test. All the experiments were performed at least twice, with triplicate skin samples.

The number of dermal mast cells detected by toluidine blue staining in UVB-irradiated Skh:hr-1 mouse skin was significantly (p<0.001) up-regulated by 45% to 14.2 cells per field at 72 h post-UVB, compared to unirradiated control mice (9.8 cells per field) as shown in FIG. 24. Cpd. 18 significantly (p<0.001) reduced the UVB-increased mast cell number to a prevalence no different from the unirradiated skin (both 10.0 cells per field).

### 3.5 Immunomodulatory Activity in Experimental Autoimmune Encephalomyelitis (EAE)

The effect of Cpd. 18 against the development of EAE in SJL/J mice immunised with the neuroantigen peptide (PLP 139-151) was examined. This is a model for multiple sclerosis in humans, and simulates the characteristic acute episode followed by repeated remission and relapse.

### Method

EAE induction was carried by immunisation with PLP 139-151 (HSLGKWLGHPDKF-NH2) in incomplete Freund's adjuvant (Sigma) supplemented with 4 mg/ml of Mycobacterium tuberculosis (strain H37Ra) as described previously, Groups of 6 female mice aged 4-5 weeks old were immunised, and body weight and the development neurological signs were monitored daily for 6 weeks. Clinical signs were scored according to an established scale:

| | |
|---|---|
| 0 | no disease |
| 1 | partially flaccid tail limp tail |
| 2 | full flaccid tail |
| 3 | tail or hind limb paralysis |
| 4 | hind and fore limb paralysis |
| 5 | moribund |

Cpd. 18 was made up to a concentration of 20 μM in a vehicle of propylene glycol-ethanol-water (1:2:1) with 0.001% DMSO). An aliquot of 100 μl either of Cpd. 18 in vehicle, or the vehicle alone, was applied to the mouse dorsum (six mice per group) for 5 days before the immunisation (with PLP 139-151) and daily post-immunisation for 48 days.

### Results

Daily body weight measurements indicated periods of weight loss that signalled development of neurological signs. The combined clinical scores for the group of mice were plotted in FIGS. 25 and 26.

In the acute phase, 12-26 days, control mice had the most severe signs. Cpd. 18 tended to reduce the severity, both acutely and again at the first relapse episode at 28-36 days. Cpd. 18 treatment produced a condition of chronic relatively stable disease.

### 4 Summary

The results show that the compounds of the subject invention display attributes important in the inhibition of inflammation, including inhibition of NFκB, COX, LO, TNFα, NO and vascular adhesion molecules, robust antioxidant activity, immunomodulatory activity, PPARγ activation and vascular activity including inhibition of vascular smooth muscle cell proliferation (VSMC), the induction of eNOS and vasodilatory activity ex situ.

In particular, the compounds of the invention have particular utility in therapeutic areas having an inflammatory component in common including atherosclerosis and peripheral artery disease, metabolic syndrome and insulin resistance, arthritis including rheumatoid arthritis, osteoarthritis and chronic back pain, inflammatory skin conditions including psoriasis and dermatitis/eczema, immunologically mediated skin conditions including pemphigus and bullous pemphigoid, ocular therapeutic areas such as ocular inflammation including allergic conjunctivitis, pre- and post-surgery eye trauma, scleritis and uveitis, macular degeneration, cataracts, keratoconjunctivitis sicca (KCS) or ‘dry eye’ and diabetic retinopathy.

The reference to any prior art in this specification is not, and should not be taken as, an acknowledgment or any form of suggestion that that prior art forms part of the common general knowledge in the field of endeavour.

Those skilled in the art will appreciate that the invention described herein is susceptible to variations and modifications other than those specifically described. It is to be understood that the invention includes all such variations and modifications. The inventions also includes all of the steps, features, compositions and compounds referred to or indicated in the specification, individually or collectively, and any and all combinations of any two or more of said steps or features.

1. A method for the treatment or prophylaxis of an inflammatory disease or a disease associated with oxidant stress which comprises the step of administering to a subject a therapeutically effective amount of one or more compounds of the general formula: 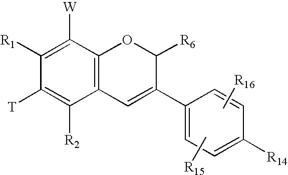
in which
R₁ is hydroxy or OC(O)R₁₀,
R₂ is hydrogen, hydroxy, OR₉, OC(O)R₁₀, alkyl or halo,
T is hydrogen, alkyl or halo,
W is hydrogen, hydroxy, OC(O)R₁₀, alkyl or halo,
R₆ is hydrogen,
R₉ is alkyl,
R₁₀ is hydrogen or alkyl,
R₁₄, R₁₅ and R₁₆ are independently hydrogen, hydroxy, OR₉, OC(O)R₁₀ or halo,
or a pharmaceutically acceptable salt thereof,
with the proviso that
when
R₁ is hydroxy or OC(O)R_{A} where R_{A} is alkyl, and
R₂ is hydrogen, hydroxy, OR_{B} where R_{B} is C(O)R_{A} where R_{A} is alkyl,
W is hydrogen, and
T is hydrogen, then
Y is not phenyl, 4-hydroxyphenyl, 4-acetoxyphenyl, 4-alkoxyphenyl or 4-alkylphenyl; and
with the proviso that the following compounds are excluded:

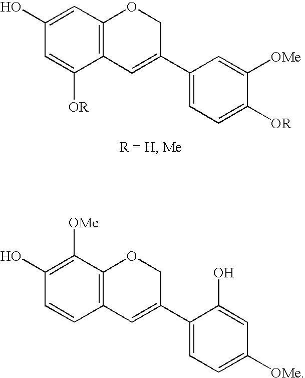

2. The method according to claim 1, wherein R₁₀ is alkyl.

3. The method according to claim 1, wherein R₁₅ is hydrogen and R₁₆ is in the 3-position.

4. The method according to claim 3, wherein R₁₄ and R₁₆ are independently hydrogen, hydroxy, methoxy or halo.

5. The method according to claim 3, wherein at least one of R₁₄ and R₁₆ is hydroxy.

6. The method according to claim 3, wherein at least one of R₁₄ and R₁₆ is methoxy.

7. The method according to claim 1, wherein one of T, W and R₂ is hydroxy, methyl, methoxy or halo.

8. The method according to claim 7, wherein one of T, W and R₂ is methyl.

9. The method according to claim 7, wherein one of T, W and R₂ is halo.

10. The method according to claim 1, wherein halo is chloro or bromo.

11. The method according to claim 1, wherein the one or more compounds are selected from:

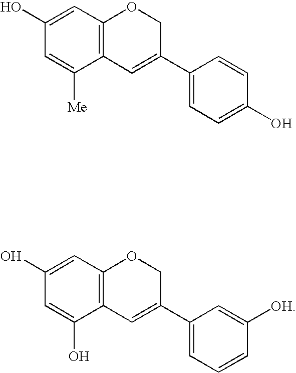

12. The method according to claim 1, wherein the disease is an inflammatory disease selected from inflammatory bowel disease, ulcerative colitis or Crohn's disease.

13. The method according to claim 1, wherein the disease is atherosclerosis or myocardial infarction.

14. The method according to claim 1, wherein the disease is a rheumatic disease or arthritis.

15. The method according to claim 1, wherein the disease is psoriasis.

16. The method according to claim 1, wherein the disease is sunlight induced skin damage.

17. The method according to claim 1, wherein the disease is cataracts.

18. The method according to claim 1, wherein the one or more compounds are administered as a topical composition.

19. The method according to claim 18, wherein the topical composition is a cosmetic formulation.

20. The method according to claim 1, wherein the one or more compounds are administered as an optical composition.
